SEQUENCE LISTING (33 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3839 bp, Artificial Sequence
  SARS-CoV2 spike protein nucleic acid - optimised for E. coli expression
gagctcatgtttgtttttctggttctgctgccgctggttagcagccagtgtgttaatctgaccacacgtacccagctgcctccggcatataccaatagctttacccgtggtgtttattatccggacaaagtttttcgtagcagcgttctgcatagcacccaggacctgtttctgccgttttttagcaatgttacctggtttcatgccattcatgttagcggcaccaatggcaccaaacgttttgataatccggtgctgccgtttaatgatggtgtgtattttgcaagcaccgaaaaaagcaacattattcgcggttggatttttggtacaaccctggatagcaaaacccagagcctgctgattgttaataatgccaccaatgtggtgatcaaagtgtgcgaatttcagttttgcaatgatccgtttctgggcgtgtattaccacaaaaataacaagagctggatggaaagcgaatttcgtgtttatagcagcgccaataattgcacctttgaatatgttagccagccgtttctgatggatctggaaggtaaacagggtaactttaaaaacctgcgcgagttcgtgttcaaaaacatcgatggttacttcaaaatctatagcaaacacaccccgattaatctggttcgtgatctgccgcagggttttagcgcactggaaccgctggttgatctgccaattggtattaacattacccgttttcagaccctgctggcactgcatcgtagctatctgacaccgggtgatagcagcagcggttggaccgcaggcgcagcagcatattatgttggttatctgcagcctcgtacctttctgctgaaatataacgaaaatggcacaattaccgatgccgttgattgtgccctggatccgctgagcgaaaccaaatgtaccctgaaaagctttaccgttgagaaaggtatttatcagaccagcaattttcgtgtgcagccgaccgaaagcattgttcgttttccgaatatcaccaatctgtgtccgtttggcgaagtttttaatgcaacccgttttgccagcgtttatgcatggaatcgtaaacgtattagcaattgcgttgccgattatagcgttctgtataatagcgcaagcttcagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactataaactgccggatgattttacgggttgtgttattgcctggaatagcaataatctggacagcaaagttggtggcaactataactatctgtatcgcctgtttcgtaagagcaatctgaaaccgtttgaacgtgatattagcaccgagatttatcaggcaggtagcaccccgtgtaatggtgttgaaggttttaattgctattttccgctgcagagctatggttttcagccgacaaatggtgtgggttatcagccgtatcgtgttgttgttctgtcatttgaactgctgcatgcaccggcaaccgtttgtggtccgaaaaaaagtaccaatctggtgaaaaataagtgcgtgaactttaactttaatggtctgaccggcaccggtgttctgaccgaaagtaacaaaaaattcctgccgtttcagcagtttggccgtgatattgcagataccaccgatgcagttcgcgatccgcagacactggaaattctggatattaccccgtgcagctttggtggtgtttcagttattacaccgggtacaaataccagcaatcaggttgcagttctgtatcaggatgttaattgtaccgaagttccggttgcaattcatgcagatcagctgaccccgacctggcgtgtgtatagcaccggtagcaatgtgtttcagacacgtgcaggttgtctgattggtgcagaacatgtgaataatagctatgaatgcgatattccgattggtgcgggtatttgtgccagctatcagacccagaccaatagtccgcgtcgtgcacgtagcgttgcaagccagagcattattgcctataccatgagcctgggtgcagaaaatagcgttgcctatagtaataacagcattgccattccgaccaactttaccattagcgttaccaccgaaattctgccggttagcatgaccaaaaccagcgttgattgcaccatgtatatttgtggtgatagtaccgaatgtagcaatctgctgctgcagtatggtagcttttgcacccagctgaatcgtgcactgaccggtattgcagttgaacaggataaaaacacgcaagaagtttttgcacaggtcaagcagatctataaaacccctccgattaaagattttggcggtttcaattttagccagatcctgccggatccgagcaaaccgagtaaacgtagctttattgaagatctgctgttcaacaaagtgaccctggcagatgcaggttttatcaaacagtatggtgattgcctgggcgatattgccgcacgtgatctgatttgtgcacagaaatttaacggcctgaccgttctgcctccgctgctgaccgatgaaatgattgcacagtataccagcgcactgctggcaggcaccattaccagtggttggacctttggtgccggtgccgcactgcagattccgtttgcaatgcagatggcatatcgttttaatggtattggtgttacccagaacgtgctgtatgaaaaccagaaactgattgccaaccagtttaatagcgccattggcaaaattcaggatagcctgagcagcaccgcaagtgcactgggtaaactgcaggacgttgttaatcagaatgcacaggcactgaataccctggttaaacagctgagcagtaattttggtgcaatttcaagcgtgctgaacgatattctgagccgtctggataaagttgaagcagaagttcagattgatcgtctgattaccggtcgtctgcaaagcctgcagacctatgtgacccagcagctgattcgcgcagcagaaattcgtgcaagcgcaaatctggcagccaccaaaatgagcgaatgtgttctgggtcagagcaaacgtgttgatttttgcggcaaaggttatcacctgatgagctttccgcagagcgcaccgcatggtgttgtgtttctgcatgttacctatgttccggcacaagaaaaaaactttacaaccgctccggcaatttgccatgatggtaaagcacattttccgcgtgaaggtgtttttgttagtaatggcacccattggtttgttacacagcgcaacttttatgaaccgcagattattacaaccgacaacacctttgttagcggtaactgtgatgttgtgattggcattgtgaataacaccgtttatgatccactgcagccggaactggatagctttaaagaagaactggacaaatatttcaaaaaccacaccagtccggatgttgatctgggtgatatttcaggtattaatgccagcgtggtgaacatccagaaagaaattgatcgcctgaatgaagtggccaaaaatctgaatgaaagcctgattgatctgcaagaactggggaaatatgagcagtatatcaaatggccgtggtatatttggctgggttttattgcaggcctgattgcaattgttatggtgaccattatgctgtgttgtatgaccagctgttgtagctgtctgaaaggttgttgcagctgcggtagctgttgcaaatttgatgaagatgatagcgaaccggtgctgaaaggtgttaaactgcattatacctaatgagcggccgc

SEQ ID NO: 3 - DNA, 4556 bp, Artificial Sequence
  nucleic acid encoding for fusion protein HEV-SARS-CoV-2 spike protein- optimised for expression in E. coli
gagctcatgattgcactgaccctgtttaatctggcagataccctgttaggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgacaccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtttaggtgcaggtccggttagcattagcgcagttgcagttctggcaccgcatagcgcatttgtttttctggttctgctgccgctggttagcagccagtgtgttaatctgaccacacgtacccagctgcctccggcatataccaatagctttacccgtggtgtttattatccggacaaagtttttcgtagcagcgttctgcatagcacccaggacctgtttctgccgttttttagcaatgttacctggtttcatgccattcatgttagcggcaccaatggcaccaaacgttttgataatccggtgctgccgtttaatgatggtgtgtattttgcaagcaccgaaaaaagcaacattattcgcggttggatttttggtacaaccctggatagcaaaacccagagcctgctgattgttaataatgccaccaatgtggtgatcaaagtgtgcgaatttcagttttgcaatgatccgtttctgggcgtgtattaccacaaaaataacaagagctggatggaaagcgaatttcgtgtttatagcagcgccaataattgcacctttgaatatgttagccagccgtttctgatggatctggaaggtaaacagggtaactttaaaaacctgcgcgagttcgtgttcaaaaacatcgatggttacttcaaaatctatagcaaacacaccccgattaatctggttcgtgatctgccgcagggttttagcgcactggaaccgctggttgatctgccaattggtattaacattacccgttttcagaccctgctggcactgcatcgtagctatctgacaccgggtgatagcagcagcggttggaccgcaggcgcagcagcatattatgttggttatctgcagcctcgtacctttctgctgaaatataacgaaaatggcacaattaccgatgccgttgattgtgccctggatccgctgagcgaaaccaaatgtaccctgaaaagctttaccgttgagaaaggtatttatcagaccagcaattttcgtgtgcagccgaccgaaagcattgttcgttttccgaatatcaccaatctgtgtccgtttggcgaagtttttaatgcaacccgttttgccagcgtttatgcatggaatcgtaaacgtattagcaattgcgttgccgattatagcgttctgtataatagcgcaagcttcagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactataaactgccggatgattttacgggttgtgttattgcctggaatagcaataatctggacagcaaagttggtggcaactataactatctgtatcgcctgtttcgtaagagcaatctgaaaccgtttgaacgtgatattagcaccgagatttatcaggcaggtagcaccccgtgtaatggtgttgaaggttttaattgctattttccgctgcagagctatggttttcagccgacaaatggtgtgggttatcagccgtatcgtgttgttgttctgtcatttgaactgctgcatgcaccggcaaccgtttgtggtccgaaaaaaagtaccaatctggtgaaaaataagtgcgtgaactttaactttaatggtctgaccggcaccggtgttctgaccgaaagtaacaaaaaattcctgccgtttcagcagtttggccgtgatattgcagataccaccgatgcagttcgcgatccgcagacactggaaattctggatattaccccgtgcagctttggtggtgtttcagttattacaccgggtacaaataccagcaatcaggttgcagttctgtatcaggatgttaattgtaccgaagttccggttgcaattcatgcagatcagctgaccccgacctggcgtgtgtatagcaccggtagcaatgtgtttcagacacgtgcaggttgtctgattggtgcagaacatgtgaataatagctatgaatgcgatattccgattggtgcgggtatttgtgccagctatcagacccagaccaatagtccgcgtcgtgcacgtagcgttgcaagccagagcattattgcctataccatgagcctgggtgcagaaaatagcgttgcctatagtaataacagcattgccattccgaccaactttaccattagcgttaccaccgaaattctgccggttagcatgaccaaaaccagcgttgattgcaccatgtatatttgtggtgatagtaccgaatgtagcaatctgctgctgcagtatggtagcttttgcacccagctgaatcgtgcactgaccggtattgcagttgaacaggataaaaacacgcaagaagtttttgcacaggtcaagcagatctataaaacccctccgattaaagattttggcggtttcaattttagccagatcctgccggatccgagcaaaccgagtaaacgtagctttattgaagatctgctgttcaacaaagtgaccctggcagatgcaggttttatcaaacagtatggtgattgcctgggcgatattgccgcacgtgatctgatttgtgcacagaaatttaacggcctgaccgttctgcctccgctgctgaccgatgaaatgattgcacagtataccagcgcactgctggcaggcaccattaccagtggttggacctttggtgccggtgccgcactgcagattccgtttgcaatgcagatggcatatcgttttaatggtattggtgttacccagaacgtgctgtatgaaaaccagaaactgattgccaaccagtttaatagcgccattggcaaaattcaggatagcctgagcagcaccgcaagtgcactgggtaaactgcaggacgttgttaatcagaatgcacaggcactgaataccctggttaaacagctgagcagtaattttggtgcaatttcaagcgtgctgaacgatattctgagccgtctggataaagttgaagcagaagttcagattgatcgtctgattaccggtcgtctgcaaagcctgcagacctatgtgacccagcagctgattcgcgcagcagaaattcgtgcaagcgcaaatctggcagccaccaaaatgagcgaatgtgttctgggtcagagcaaacgtgttgatttttgcggcaaaggttatcacctgatgagctttccgcagagcgcaccgcatggtgttgtgtttctgcatgttacctatgttccggcacaagaaaaaaactttacaaccgctccggcaatttgccatgatggtaaagcacattttccgcgtgaaggtgtttttgttagtaatggcacccattggtttgttacacagcgcaacttttatgaaccgcagattattacaaccgacaacacctttgttagcggtaactgtgatgttgtgattggcattgtgaataacaccgtttatgatccactgcagccggaactggatagctttaaagaagaactggacaaatatttcaaaaaccacaccagtccggatgttgatctgggtgatatttcaggtattaatgccagcgtggtgaacatccagaaagaaattgatcgcctgaatgaagtggccaaaaatctgaatgaaagcctgattgatctgcaagaactggggaaatatgagcagtatatcaaatggccgtggtatatttggctgggttttattgcaggcctgattgcaattgttatggtgaccattatgctgtgttgtatgaccagctgttgtagctgtctgaaaggttgttgcagctgcggtagctgttgcaaatttgatgaagatgatagcgaaccggtgctgaaaggtgttaaactgcattatacctaatgagcggccgc

SEQ ID NO: 4 - DNA, 3839 bp, Artificial Sequence
  SARS-CoV-2 spike protein nucleic acid sequence - optimised for expression in Komagataella pastoris
ttcgaaacgatgttcgtgttcttggtcctgttgccattggtttcttcccagtgtgttaacctgaccactagaactcaattgcctccagcctacaccaattccttcaccagaggtgtttactacccagacaaggtgttcagatcttccgtcttgcactccactcaggacttgttcttgccattcttctccaacgttacctggttccacgctattcacgtttccggaactaacggtactaagagattcgacaacccagtcctgccattcaacgatggtgtctacttcgcttctaccgagaagtccaacatcatcagaggttggatcttcggtactaccctggactctaagactcagtccttgctgatcgttaacaacgccaccaacgttgtcatcaaggtttgcgagttccagttctgcaacgacccattcttgggtgtgtactaccacaagaacaacaagtcttggatggaatccgagttcagagtttactcctccgccaacaactgtaccttcgagtacgtttcccagccattcttgatggacttggagggtaagcagggtaacttcaagaacctgagagagttcgttttcaagaacatcgacggttacttcaagatctactccaagcacaccccaatcaacctggttagagatttgccacaaggtttctccgctttggagcctttggttgacttgccaatcggtatcaacatcaccagattccagaccttgttggccttgcacagatcctacttgactccaggtgattcttcttccggttggactgctggtgctgctgcttactatgttggttacttgcagccaagaaccttcctgctgaagtacaacgagaacggaactatcactgacgctgttgactgtgctttggacccattgtctgagactaagtgcaccttgaagtccttcaccgttgagaagggtatctaccagacctccaacttcagagttcagccaactgagtccatcgtcagattcccaaacatcactaacttgtgcccattcggtgaggtgttcaacgctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacttgaagccattcgagagagacatctccaccgagatctaccaagctggttctactccatgtaacggtgtcgagggtttcaactgctacttcccattgcaatcctacggtttccaacctaccaacggtgttggataccagccatacagagttgtcgttttgtccttcgagttgttgcacgctccagctactgtttgtggtccaaagaagtccaccaacttggtcaagaacaaatgcgtcaactttaacttcaacggcctgaccggtactggtgttttgactgaatccaacaagaagttcctgcctttccagcagttcggtagagacattgctgacactactgacgccgttagagatccacagactttggagatcttggacatcaccccatgttccttcggtggtgtttccgttattacccctggaactaacacctccaatcaggtcgctgtcttgtaccaggacgttaactgtactgaggttccagttgctatccacgctgaccaattgactccaacttggagagtctactccaccggttccaacgttttccaaactagagccggttgtttgatcggtgctgaacacgtcaacaactcctacgagtgtgacattccaattggtgctggtatctgtgcctcctaccaaactcaaactaactccccaagaagggctagatccgttgcttcccaatccattatcgcttacaccatgtctttgggtgccgagaactctgttgcctactctaacaactctatcgctatccctaccaacttcaccatctccgttaccactgagatcttgccagtctccatgaccaagacttccgttgactgtaccatgtacatctgtggtgactccactgagtgttccaacttgttgctgcaatacggttccttctgcacccagttgaacagagctttgactggtattgctgtcgagcaagacaagaacactcaagaggttttcgcccaggtgaagcagatctacaagactccacctattaaggacttcggtggcttcaacttctcccagattttgccagatccatctaagccctccaagagatccttcattgaggacctgctgttcaacaaggttactttggctgacgccggtttcatcaagcagtacggtgattgcttgggtgacattgcagctagagacttgatctgtgcccagaagttcaacggtttgaccgttttgccacctttgttgaccgacgagatgatcgctcagtacacttctgctttgttggccggtactatcacttctggttggacatttggagctggtgccgcattgcaaattccattcgctatgcaaatggcctacagattcaacggtatcggtgttacccagaacgtcctgtacgagaaccagaagcttatcgccaaccagttcaactccgctatcggtaagattcaggactccttgtcctctactgcttctgccttgggaaagttgcaggatgttgttaaccagaatgcccaggctttgaacaccctggttaagcaactgtcctctaacttcggtgctatctcctccgttttgaacgacatcttgtcccgtttggacaaggttgaggctgaggttcagatcgacagattgatcactggtagattgcagtccctgcagacttacgttactcagcagttgattagagctgccgagattagagcctctgctaacttggctgctactaagatgtccgagtgtgttttgggtcagtccaagagagttgacttctgcggtaagggttaccacctgatgtctttcccacaatctgctccacacggtgtcgttttcttgcacgttacttacgttccagctcaagagaagaacttcactactgctccagccatttgtcacgatggtaaggctcactttcctcgtgagggtgttttcgtttccaacggtactcactggttcgtcacccagagaaacttttacgagccacagatcatcaccaccgacaacactttcgtttctggtaactgtgacgtcgtcatcggtatcgtgaacaacactgtctacgatccattgcagccagaattggactccttcaaagaggaactggacaagtactttaagaaccacacttccccagacgttgacctgggtgatatttccggtattaacgcctccgttgtcaacatccaaaaagagatcgaccgtttgaacgaggtcgccaagaacttgaacgagtccttgattgacttgcaagagctgggcaagtacgagcagtacattaagtggccatggtacatttggctgggtttcattgctggtttgatcgccatcgttatggtcaccatcatgttgtgctgtatgacctcctgttgctcctgtttgaagggttgttgttcctgcggttcctgttgtaagttcgacgaagatgactccgagccagtcttgaagggtgttaagttgcactacacttaagcggccgc

SEQ ID NO: 5 - DNA, 5357 bp, Artificial Sequence
  nucleic acid encoding for fusion protein HPV18L1/SARS-CoV-2 spike protein- optimised for expression in K. pastoris
ttcgaaacgatggctctttggagaccatccgacaacactgtttacttgccaccaccatccgttgctagagttgttaacactgacgactacgttactagaacttccatcttctaccacgctggttcttccagattgttgactgttggtaacccatacttcagagttccagctggaggtggtaacaagcaagacatcccaaaggtttccgcttaccagtacagagttttcagagttcagttgccagacccaaacaagtttggattgccagacacttccatctacaacccagagactcagagacttgtttgggcttgtgctggtgttgaaatcggtagaggacagccattgggtgttggtttgtctggtcacccattctacaacaagttggacgacactgaatcttctcacgctgctacttctaacgtttccgaggatgttagagacaacgtttccgttgactacaagcagactcagttgtgtatcttgggttgtgctccagctattggtgaacattgggctaagggtactgcttgtaagtccagaccattgtctcagggagattgtccaccattggagttgaagaacactgttttggaggacggtgatatggttgatactggttacggtgctatggacttctctactttgcaggacactaagtgtgaagttccattggacatctgtcagtccatctgtaagtacccagactacttgcaaatgtccgctgatccatacggtgactctatgttcttctgtttgagaagagagcagttgttcgctagacacttctggaacagagctggtactatgggtgacactgttccacaatccttgtacatcaagggtactggaatgagagcttctcctggttcttgtgtttactctccatctccatccggttccattgttacttccgactcccagttgttcaacaagccatactggttgcataaggctcaaggtcacaacaacggtgtttgttggcacaaccagttgttcgttactgttgttgacactactagatccactaacttgactatctgtgcttccactcaatctccagttccaggacaatacgacgctactaagttcaagcagtactccagacacgttgaagagtacgacttgcagttcatcttccagttgtgtactatcactttgactgctgatgttatgtcctacatccactctatgaactcctccattttggaggattggaacttcggtgttccaccaccaccaactacttcattggttgacacttacagattcgttcagtccgttgctatcacttgtcaaaaggacgctgctccagctgaaaacaaggacccatacgacaagttgaagttctggaacgttgacttgaaagagaagttctccttggacttggaccaatacccattgggtagaaagtttttggttcaggctggattgagaagaaagccaactatcggtccaagaaagagatcagctccatccgctactacttcatccaagccagctaagagagttagagttagagctagaaagttcgtgttcttggtcctgttgccattggtttcttcccagtgtgttaacctgaccactagaactcaattgcctccagcctacaccaattccttcaccagaggtgtttactacccagacaaggtgttcagatcttccgtcttgcactccactcaggacttgttcttgccattcttctccaacgttacctggttccacgctattcacgtttccggaactaacggtactaagagattcgacaacccagtcctgccattcaacgatggtgtctacttcgcttctaccgagaagtccaacatcatcagaggttggatcttcggtactaccctggactctaagactcagtccttgctgatcgttaacaacgccaccaacgttgtcatcaaggtttgcgagttccagttctgcaacgacccattcttgggtgtgtactaccacaagaacaacaagtcttggatggaatccgagttcagagtttactcctccgccaacaactgtaccttcgagtacgtttcccagccattcttgatggacttggagggtaagcagggtaacttcaagaacctgagagagttcgttttcaagaacatcgacggttacttcaagatctactccaagcacaccccaatcaacctggttagagatttgccacaaggtttctccgctttggagcctttggttgacttgccaatcggtatcaacatcaccagattccagaccttgttggccttgcacagatcctacttgactccaggtgattcttcttccggttggactgctggtgctgctgcttactatgttggttacttgcagccaagaaccttcctgctgaagtacaacgagaacggaactatcactgacgctgttgactgtgctttggacccattgtctgagactaagtgcaccttgaagtccttcaccgttgagaagggtatctaccagacctccaacttcagagttcagccaactgagtccatcgtcagattcccaaacatcactaacttgtgcccattcggtgaggtgttcaacgctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacttgaagccattcgagagagacatctccaccgagatctaccaagctggttctactccatgtaacggtgtcgagggtttcaactgctacttcccattgcaatcctacggtttccaacctaccaacggtgttggataccagccatacagagttgtcgttttgtccttcgagttgttgcacgctccagctactgtttgtggtccaaagaagtccaccaacttggtcaagaacaaatgcgtcaactttaacttcaacggcctgaccggtactggtgttttgactgaatccaacaagaagttcctgcctttccagcagttcggtagagacattgctgacactactgacgccgttagagatccacagactttggagatcttggacatcaccccatgttccttcggtggtgtttccgttattacccctggaactaacacctccaatcaggtcgctgtcttgtaccaggacgttaactgtactgaggttccagttgctatccacgctgaccaattgactccaacttggagagtctactccaccggttccaacgttttccaaactagagccggttgtttgatcggtgctgaacacgtcaacaactcctacgagtgtgacattccaattggtgctggtatctgtgcctcctaccaaactcaaactaactccccaagaagggctagatccgttgcttcccaatccattatcgcttacaccatgtctttgggtgccgagaactctgttgcctactctaacaactctatcgctatccctaccaacttcaccatctccgttaccactgagatcttgccagtctccatgaccaagacttccgttgactgtaccatgtacatctgtggtgactccactgagtgttccaacttgttgctgcaatacggttccttctgcacccagttgaacagagctttgactggtattgctgtcgagcaagacaagaacactcaagaggttttcgcccaggtgaagcagatctacaagactccacctattaaggacttcggtggcttcaacttctcccagattttgccagatccatctaagccctccaagagatccttcattgaggacctgctgttcaacaaggttactttggctgacgccggtttcatcaagcagtacggtgattgcttgggtgacattgcagctagagacttgatctgtgcccagaagttcaacggtttgaccgttttgccacctttgttgaccgacgagatgatcgctcagtacacttctgctttgttggccggtactatcacttctggttggacatttggagctggtgccgcattgcaaattccattcgctatgcaaatggcctacagattcaacggtatcggtgttacccagaacgtcctgtacgagaaccagaagcttatcgccaaccagttcaactccgctatcggtaagattcaggactccttgtcctctactgcttctgccttgggaaagttgcaggatgttgttaaccagaatgcccaggctttgaacaccctggttaagcaactgtcctctaacttcggtgctatctcctccgttttgaacgacatcttgtcccgtttggacaaggttgaggctgaggttcagatcgacagattgatcactggtagattgcagtccctgcagacttacgttactcagcagttgattagagctgccgagattagagcctctgctaacttggctgctactaagatgtccgagtgtgttttgggtcagtccaagagagttgacttctgcggtaagggttaccacctgatgtctttcccacaatctgctccacacggtgtcgttttcttgcacgttacttacgttccagctcaagagaagaacttcactactgctccagccatttgtcacgatggtaaggctcactttcctcgtgagggtgttttcgtttccaacggtactcactggttcgtcacccagagaaacttttacgagccacagatcatcaccaccgacaacactttcgtttctggtaactgtgacgtcgtcatcggtatcgtgaacaacactgtctacgatccattgcagccagaattggactccttcaaagaggaactggacaagtactttaagaaccacacttccccagacgttgacctgggtgatatttccggtattaacgcctccgttgtcaacatccaaaaagagatcgaccgtttgaacgaggtcgccaagaacttgaacgagtccttgattgacttgcaagagctgggcaagtacgagcagtacattaagtggccatggtacatttggctgggtttcattgctggtttgatcgccatcgttatggtcaccatcatgttgtgctgtatgacctcctgttgctcctgtttgaagggttgttgttcctgcggttcctgttgtaagttcgacgaagatgactccgagccagtcttgaagggtgttaagttgcactacacttaagcggccgc

SEQ ID NO: 6 - DNA, 5351 bp, Artificial Sequence
  nucleic acid encoding for fusion protein HPV16L1/SARS-CoV-2 spike protein - optimised for expression in K. pastoris
ttcgaaacgatgtctttgtggttgccatctgaagctactgtttacttgccaccagttccagtttctaaagttgtttccactgacgaatacgttgctagaactaacatctactaccacgctggtacttctagattgttggctgttggtcatccatacttcccaattaagaagccaaacaacaacaagattttggttccaaaggtttccggattgcaatacagagttttcagaatccatttgccagatccaaacaagtttggtttcccagatacttctttctacaacccagacactcaaagacttgtttgggcttgtgttggtgttgaagttggtagaggtcaaccattgggtgttggtatttctggtcacccattgttgaacaagttggacgatactgaaaacgcttctgcttacgctgctaacgctggtgttgataacagagaatgtatttctatggactacaagcaaactcaattgtgtttgattggttgtaagccaccaattggtgaacattggggaaagggttctccatgtactaatgttgctgttaaccctggtgattgtccaccattggaattgattaacactgttattcaagacggtgatatggttgatactggtttcggtgctatggatttcactactttgcaagctaacaagtctgaagttccattggacatttgtacttccatctgtaagtacccagactacattaagatggtttctgaaccatacggtgattctttgttcttctacttgagaagagaacaaatgtttgttagacacttgttcaacagagctggtgctgttggtgaaaacgttccagatgacttgtacattaagggttctggttctactgctaacttggcttcttctaactactttccaactccatctggttctatggttacttctgacgctcaaattttcaacaagccatactggttgcaaagagcacaaggtcataacaacggtatttgttggggtaaccaattgttcgttactgttgttgacactactagatccactaacatgtccttgtgtgctgctatttctacttctgaaactacttacaagaacactaacttcaaagagtacttgagacacggagaagaatacgacttgcaattcattttccaattgtgtaagattactttgactgctgacgttatgacttacattcactctatgaactctactattttggaagattggaacttcggattgcaaccaccaccaggtggtactttggaagatacttacagattcgttacttctcaagctattgcttgtcaaaagcatactccacctgctccaaaagaagatccattgaagaagtacactttctgggaagttaacttgaaagaaaagttctctgctgatttggatcaattcccattgggtagaaagtttttgttgcaagctggattgaaggctaaaccaaagttcactttgggaaagagaaaggctactccaactacttcttctacttctactactgctaagagaaagaagagaaaattgttcgtgttcttggtcctgttgccattggtttcttcccagtgtgttaacctgaccactagaactcaattgcctccagcctacaccaattccttcaccagaggtgtttactacccagacaaggtgttcagatcttccgtcttgcactccactcaggacttgttcttgccattcttctccaacgttacctggttccacgctattcacgtttccggaactaacggtactaagagattcgacaacccagtcctgccattcaacgatggtgtctacttcgcttctaccgagaagtccaacatcatcagaggttggatcttcggtactaccctggactctaagactcagtccttgctgatcgttaacaacgccaccaacgttgtcatcaaggtttgcgagttccagttctgcaacgacccattcttgggtgtgtactaccacaagaacaacaagtcttggatggaatccgagttcagagtttactcctccgccaacaactgtaccttcgagtacgtttcccagccattcttgatggacttggagggtaagcagggtaacttcaagaacctgagagagttcgttttcaagaacatcgacggttacttcaagatctactccaagcacaccccaatcaacctggttagagatttgccacaaggtttctccgctttggagcctttggttgacttgccaatcggtatcaacatcaccagattccagaccttgttggccttgcacagatcctacttgactccaggtgattcttcttccggttggactgctggtgctgctgcttactatgttggttacttgcagccaagaaccttcctgctgaagtacaacgagaacggaactatcactgacgctgttgactgtgctttggacccattgtctgagactaagtgcaccttgaagtccttcaccgttgagaagggtatctaccagacctccaacttcagagttcagccaactgagtccatcgtcagattcccaaacatcactaacttgtgcccattcggtgaggtgttcaacgctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacttgaagccattcgagagagacatctccaccgagatctaccaagctggttctactccatgtaacggtgtcgagggtttcaactgctacttcccattgcaatcctacggtttccaacctaccaacggtgttggataccagccatacagagttgtcgttttgtccttcgagttgttgcacgctccagctactgtttgtggtccaaagaagtccaccaacttggtcaagaacaaatgcgtcaactttaacttcaacggcctgaccggtactggtgttttgactgaatccaacaagaagttcctgcctttccagcagttcggtagagacattgctgacactactgacgccgttagagatccacagactttggagatcttggacatcaccccatgttccttcggtggtgtttccgttattacccctggaactaacacctccaatcaggtcgctgtcttgtaccaggacgttaactgtactgaggttccagttgctatccacgctgaccaattgactccaacttggagagtctactccaccggttccaacgttttccaaactagagccggttgtttgatcggtgctgaacacgtcaacaactcctacgagtgtgacattccaattggtgctggtatctgtgcctcctaccaaactcaaactaactccccaagaagggctagatccgttgcttcccaatccattatcgcttacaccatgtctttgggtgccgagaactctgttgcctactctaacaactctatcgctatccctaccaacttcaccatctccgttaccactgagatcttgccagtctccatgaccaagacttccgttgactgtaccatgtacatctgtggtgactccactgagtgttccaacttgttgctgcaatacggttccttctgcacccagttgaacagagctttgactggtattgctgtcgagcaagacaagaacactcaagaggttttcgcccaggtgaagcagatctacaagactccacctattaaggacttcggtggcttcaacttctcccagattttgccagatccatctaagccctccaagagatccttcattgaggacctgctgttcaacaaggttactttggctgacgccggtttcatcaagcagtacggtgattgcttgggtgacattgcagctagagacttgatctgtgcccagaagttcaacggtttgaccgttttgccacctttgttgaccgacgagatgatcgctcagtacacttctgctttgttggccggtactatcacttctggttggacatttggagctggtgccgcattgcaaattccattcgctatgcaaatggcctacagattcaacggtatcggtgttacccagaacgtcctgtacgagaaccagaagcttatcgccaaccagttcaactccgctatcggtaagattcaggactccttgtcctctactgcttctgccttgggaaagttgcaggatgttgttaaccagaatgcccaggctttgaacaccctggttaagcaactgtcctctaacttcggtgctatctcctccgttttgaacgacatcttgtcccgtttggacaaggttgaggctgaggttcagatcgacagattgatcactggtagattgcagtccctgcagacttacgttactcagcagttgattagagctgccgagattagagcctctgctaacttggctgctactaagatgtccgagtgtgttttgggtcagtccaagagagttgacttctgcggtaagggttaccacctgatgtctttcccacaatctgctccacacggtgtcgttttcttgcacgttacttacgttccagctcaagagaagaacttcactactgctccagccatttgtcacgatggtaaggctcactttcctcgtgagggtgttttcgtttccaacggtactcactggttcgtcacccagagaaacttttacgagccacagatcatcaccaccgacaacactttcgtttctggtaactgtgacgtcgtcatcggtatcgtgaacaacactgtctacgatccattgcagccagaattggactccttcaaagaggaactggacaagtactttaagaaccacacttccccagacgttgacctgggtgatatttccggtattaacgcctccgttgtcaacatccaaaaagagatcgaccgtttgaacgaggtcgccaagaacttgaacgagtccttgattgacttgcaagagctgggcaagtacgagcagtacattaagtggccatggtacatttggctgggtttcattgctggtttgatcgccatcgttatggtcaccatcatgttgtgctgtatgacctcctgttgctcctgtttgaagggttgttgttcctgcggttcctgttgtaagttcgacgaagatgactccgagccagtcttgaagggtgttaagttgcactacacttaagcggccgc

SEQ ID NO: 7 - DNA, 3836 bp, Artificial Sequence
  SARS-CoV-2 spike protein nucleic acid sequence - optimised for expression in humans
gctagcgacatgttcgtgtttctggtgctgctgcctctggtgtccagccagtgtgtgaacctgaccaccagaacacagctgcctccagcctacaccaatagcttcaccaggggcgtgtactaccccgacaaggtgttcagatctagcgtgctgcacagcacccaggacctgtttctgcccttcttcagcaacgtgacctggttccacgccatccacgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggagtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgcaccctgaagtccttcaccgtggaaaagggcatctaccagaccagcaacttcagagtgcagcccaccgagagcatcgtgcggttccccaatatcaccaatctgtgccccttcggcgaggtgttcaatgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgcccgtggccattcacgccgatcaactgacacccacttggagagtgtactccaccggctccaacgtgttccagactagagccggatgtctgatcggagccgagcacgtgaacaatagctacgagtgcgacatccccatcggcgctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagctttatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagctggcgcagccctgcagatcccctttgctatgcagatggcctaccggttcaacggcatcggagtgacccagaatgtgctgtacgagaaccagaagctgatcgccaaccagttcaacagcgccatcggcaagatccaggatagcctgtctagcacagccagcgctctgggcaaactgcaggacgtggtcaatcagaacgctcaggccctgaacaccctcgtgaagcagctgagcagcaatttcggcgccatcagctccgtgctgaacgatatcctgagccggctggataaggtggaagccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggatttctgcggcaagggctaccacctgatgagctttccacagtctgctcctcacggcgtggtgtttctgcacgtgacctatgtgcccgctcaagagaagaacttcacaacagcccctgccatctgccacgacggaaaggcccattttcctagagaaggcgtgttcgtgtccaacggcacccattggttcgtgacacagcggaacttctacgagccccagatcatcaccaccgacaacaccttcgtgtctggcaactgtgacgtcgtgatcggcattgtgaacaacaccgtgtacgaccctctgcagcccgagctggacagcttcaaagaggaactggacaagtactttaagaaccacacaagccccgacgtggacctgggcgatattagcggcatcaatgcctccgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgctgtagctgcctgaagggctgttgcagctgtggcagctgctgcaagttcgacgaggatgatagcgagcctgtgctgaagggcgtgaaactgcactacaccgcggccgc

SEQ ID NO: 8 - DNA, 4232 bp, Artificial Sequence
  nucleic acid encoding for fusion protein HBSAg/SARS-CoV-2 spike protein- optimised for expression in humans
gctagcgacatgaactttctgggcggtacgacagtatgccttggacaaaattcacaatctccgacgtctaatcactcccctacaagttgtccaccgacttgccccggctataggtggatgtgtctcagacgattcataatctttctcttcattcttcttctgtgcctgatattcttgctggtccttctggattaccagggaatgcttcccgtgtgtcctctgattcctggttcatccactacatctacgggtccctgtagaacatgcaccacacctgcacagggcacctccatgtatccgtcatgctgctgcacgaaaccatcagatggtaactgcacgtgcataccgatcccctcatcatgggcgtttgggaaatttctgtgggagtgggcctcagcccggttttccttcgtgtttctggtgctgctgcctctggtgtccagccagtgtgtgaacctgaccaccagaacacagctgcctccagcctacaccaatagcttcaccaggggcgtgtactaccccgacaaggtgttcagatctagcgtgctgcacagcacccaggacctgtttctgcccttcttcagcaacgtgacctggttccacgccatccacgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggagtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgcaccctgaagtccttcaccgtggaaaagggcatctaccagaccagcaacttcagagtgcagcccaccgagagcatcgtgcggttccccaatatcaccaatctgtgccccttcggcgaggtgttcaatgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgcccgtggccattcacgccgatcaactgacacccacttggagagtgtactccaccggctccaacgtgttccagactagagccggatgtctgatcggagccgagcacgtgaacaatagctacgagtgcgacatccccatcggcgctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagctttatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagctggcgcagccctgcagatcccctttgctatgcagatggcctaccggttcaacggcatcggagtgacccagaatgtgctgtacgagaaccagaagctgatcgccaaccagttcaacagcgccatcggcaagatccaggatagcctgtctagcacagccagcgctctgggcaaactgcaggacgtggtcaatcagaacgctcaggccctgaacaccctcgtgaagcagctgagcagcaatttcggcgccatcagctccgtgctgaacgatatcctgagccggctggataaggtggaagccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggatttctgcggcaagggctaccacctgatgagctttccacagtctgctcctcacggcgtggtgtttctgcacgtgacctatgtgcccgctcaagagaagaacttcacaacagcccctgccatctgccacgacggaaaggcccattttcctagagaaggcgtgttcgtgtccaacggcacccattggttcgtgacacagcggaacttctacgagccccagatcatcaccaccgacaacaccttcgtgtctggcaactgtgacgtcgtgatcggcattgtgaacaacaccgtgtacgaccctctgcagcccgagctggacagcttcaaagaggaactggacaagtactttaagaaccacacaagccccgacgtggacctgggcgatattagcggcatcaatgcctccgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgctgtagctgcctgaagggctgttgcagctgtggcagctgctgcaagttcgacgaggatgatagcgagcctgtgctgaagggcgtgaaactgcactacaccgcggccgc

SEQ ID NO: 9 - protein, 1512 aa, Artificial Sequence
  fusion protein HEV-SARS-CoV-2 spike protein
MIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTKAGYPYNYNTTASDQLLVENAAGHRVAISTYTTSLGAGPVSISAVAVLAPHSAFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1779 aa, Artificial Sequence
  fusion protein HPV18L1/SARS-CoV-2 spike protein
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARKFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1777 aa, Artificial Sequence
  fusion protein HPV16L1/SARS-CoV-2 spike protein
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKLFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1407 aa, Artificial Sequence
  fusion protein HBSAg/SARS-CoV-2 spike protein
MNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTAA

SEQ ID NO: 13 - DNA, 665 bp, Artificial Sequence
  RBD SARS-CoV-2 spike protein nucleic acid
gctagcgacgccaccatgagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgataagcggccgc

SEQ ID NO: 14 - DNA, 665 bp, Artificial Sequence
  RBD SARS-CoV-2 spike protein nucleic acid sequence - human codon optimized
gctagcgacgccaccatgagagtgcagcctacagagtctatcgtgcggttccccaacatcaccaatctgtgccctttcggcgaggtgttcaacgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggacagcaaagtcggcggcaactacaactacctgtaccggctgttccggaagtccaacctgaagcctttcgagcgggacatcagcaccgagatctatcaggccggcagcaccccttgtaatggcgtggaaggcttcaactgctacttcccactgcagtcctacggcttccagcctacaaacggcgtgggctaccagccttatagagtggtggtgctgagcttcgaactgctgcatgcccctgctacagtgtgcggccccaagaagtgataagcggccgc

SEQ ID NO: 15 - protein, 212 aa, SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 16 - protein, 7 aa, Artificial Sequence
  EAAAK linker consensus sequence
AEAAAKA

SEQ ID NO: 17 - DNA, 45 bp, Artificial Sequence
  (EAAAK)3 linker
gaagccgccgctaaagaggccgctgccaaagaagctgctgctaag

SEQ ID NO: 18 - protein, 15 aa, Artificial Sequence
  (EAAAK)3 linker
EAAAKEAAAKEAAAK

SEQ ID NO: 19 - protein, 5 aa, Artificial Sequence
  GS linker consensus sequence
GGGGS

SEQ ID NO: 20 - protein, 5 aa, Artificial Sequence
  GS5 linker
GGGGS

SEQ ID NO: 21 - protein, 10 aa, Artificial Sequence
  GS10 linker
GGGGSGGGGS

SEQ ID NO: 22 - DNA, 45 bp, Artificial Sequence
  GS15 linker
ggtggtggtggtagcggtggtggcggttcaggtggcggtggttca

SEQ ID NO: 23 - protein, 15 aa, Artificial Sequence
  GS15 linker
GGGGSGGGGSGGGGS

SEQ ID NO: 24 - protein, 20 aa, Artificial Sequence
  GS20 linker
GGGGSGGGGSGGGGSGGGGS

SEQ ID NO: 25 - protein, 25 aa, Artificial Sequence
  GS25 linker
GGGGSGGGGSGGGGSGGGGSGGGGS

SEQ ID NO: 26 - DNA, 1427 bp, Artificial Sequence
  HBSAg-(EAAAK)3-RBD
gctagcgacgccaccatgattgcactgaccctgtttaatctggcagataccctgttaggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgacaccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtttaggtgcaggtccggttagcattagcgcagttgcagttctggcaccgcattcagccgaagcagccgctaaagaagcagccgctaaagaagcagccgctaaaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgataagcggccgc

SEQ ID NO: 27 - DNA, 1106 bp, Artificial Sequence
  HBSAg-(EAAAK)3-RBD nucleic acid sequence human codon optimised
gctagcgacgccaccatgaattttctcggcggcacaacagtgtgcctgggccagaatagccagtctcctaccagcaatcacagccccaccagctgtcctccaacctgtcctggctacagatggatgtgcctgcggcggttcatcatctttctgttcatcctgctgctgtgcctgatcttcctgctggtgctgctggattaccagggaatgctgcctgtgtgtcctctgatccctggcagcagcacaacaagcacaggcccttgcagaacctgcacaacaccagctcagggcaccagcatgtaccctagctgctgttgtaccaagcctagcgacggcaactgcacatgcatccccattcctagcagctgggccttcggcaagtttctgtgggaatgggccagcgccagattttccgaagccgccgctaaagaggccgctgccaaagaagctgctgctaagagagtgcagcccaccgagtctatcgtgcggttccccaacatcaccaatctgtgccctttcggcgaggtgttcaacgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggacagcaaagtcggcggcaactacaactacctgtaccggctgttccggaagtccaacctgaagcctttcgagcgggacatcagcaccgaaatctaccaggccggcagcaccccttgtaatggcgtggaaggcttcaactgctacttcccactgcagtcctacggcttccagcctacaaacggcgtgggctaccagccttatagagtggtggtgctgagcttcgaactgctgcatgcccctgctacagtgtgcggccccaagaagtgataagcggccgc

SEQ ID NO: 28 - protein, 359 aa, Artificial Sequence
  HBSAg-(EAAAK)3-RBD
MNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSEAAAKEAAAKEAAAKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 29 - DNA, 1418 bp, Artificial Sequence
  HEV-GS15-RBD
gagctcatgattgcactgaccctgtttaatctggcagataccctgctgggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgaccccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtctgggtgcaggtccggttagcattagcgcagttgcagttctggcaccgcatagcgcaggtggaggaggttctggaggcggtggaagtggtggcggaggtagcagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgataagcggccgc

SEQ ID NO: 30 - DNA, 1418 bp, Artificial Sequence
  HEV-GS15-RBD nucleic acid sequence optimized for E.coli expression
gagctcatgattgcactgaccctgtttaatctggcagataccctgttaggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgacaccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtttaggtgcaggtccggttagcattagcgcagttgcagttctggcaccgcattcagccggtggtggtggtagcggtggtggcggttcaggtggcggtggttcacgtgttcagccgacagaaagcattgttcgttttccgaatatcaccaatctgtgtccgtttggcgaagtttttaatgcaacccgttttgcaagcgtttatgcctggaatcgtaaacgtattagcaattgcgttgccgattatagcgtgctgtataatagcgcaagctttagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactacaaactgccggatgattttacgggttgtgttattgcatggaatagcaataacctggatagcaaagttggtggcaactataactatctgtatcgcctgtttcgtaagagcaatctgaaaccgtttgaacgtgatattagcaccgaaatttatcaggcaggtagcaccccgtgcaatggtgttgaaggttttaattgttattttccgctgcagagctatggttttcagcctaccaatggtgtgggttatcagccgtatcgtgttgttgttctgtcatttgaactgctgcatgcaccggcaaccgtttgtggtccgaaaaaatgataagcggccgc

SEQ ID NO: 31 - protein, 466 aa, Artificial Sequence
  HEV-GS15-RBD
MIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTKAGYPYNYNTTASDQLLVENAAGHRVAISTYTTSLGAGPVSISAVAVLAPHSAGGGGSGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 32 - DNA, 4566 bp, Artificial Sequence
  HBSAg-(EAAAK)3-full-length SARS-CoV-2 spike protein nucleic acid sequence human codon optimised
aagcttgccgccaccatggagaacatcacatcaggattcctaggacccctgctcgtgttacaggcggggtttttcttgttgacaagaatcctcacaataccacagagtctagactcgtggtggacttctctcaattttctagggggatcacccgtgtgtctgggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccagggtatgttgcccgtttgtcctctaattccaggatcaacaactaccaacacgggaccatgcaagacctgcacgactcctgctcaaggaaactctatgtttccctcttgttgctgtacaaaacctaccgacggaaactgcacttgtattcccatcccatcatcctgggctttcgcaaaatacctatgggagtgggcctcagtccgtttctcctggctcagtttactagtgccatttgttcagtggttcgtagggctttcccccactgtttggctttccgctatatggatgatgtggtattgggggccaagtctgtacagcatcgtgagtccctttatacctctattaccaattttcttttgtctttgggtatacattgaggctgccgcaaaggaagccgcagctaaagaggcagctgccaagttcgtgttcctggttctgctgcccctggtgtctagccagtgcgtgaacctgaccaccagaacacagctgcctccagcctacaccaacagcttcaccagaggcgtgtactaccccgacaaggtgttccggtcctccgtgctgcattctacccaggacctgttcctgcctttcttctccaacgtgacctggttccacgccatccatgtgtctggcaccaacggcaccaagagattcgacaaccccgtgctgcctttcaacgacggggtgtactttgcctccaccgagaagtccaacatcatcagaggctggatcttcggcacaaccctggacagcaagacccagagcctgctgatcgtgaacaacgccaccaacgtggtcatcaaagtgtgcgagttccagttctgcaacgaccccttcctgggcgtctactaccacaagaacaacaagtcctggatggaatccgagttccgggtgtactcctccgccaacaactgcaccttcgaatacgtgtcccagcctttcctgatggacctggaaggcaagcagggcaacttcaagaacctgcgcgagttcgtgttcaagaacatcgacggctacttcaagatctactccaagcacacccctatcaacctcgtgcgggatctgcctcagggcttctctgctctggaacccctggtggatctgcccatcggcatcaacatcacccggtttcagaccctgctggccctgcaccggtcttatttgacccctggcgactcctcttctggctggactgctggcgccgctgcttactatgtgggctacctgcagcctcggacctttctgctgaagtacaacgagaatggcaccatcaccgacgccgtggactgtgctctggatcctctgtccgagacaaagtgcaccctgaagtccttcaccgtggaaaagggcatctaccagacctccaacttccgggtgcagcccaccgagtctatcgtgcggttccctaacatcaccaacctgtgtcctttcggcgaggtgttcaatgccaccagattcgcctctgtgtacgcctggaaccggaagcggatctctaactgcgtggccgactacagcgtgctgtacaactccgcctccttcagcaccttcaagtgctacggcgtgtcccctacaaagctgaacgacctgtgcttcacaaacgtgtacgccgacagcttcgtgatccggggagatgaagtgcggcagatcgctcctggacagaccggcaagatcgccgattacaactacaagctgcccgacgacttcaccggctgtgtgatcgcttggaactccaacaacctggactccaaagtcggcggcaactacaactacctgtaccggctgttccggaagtctaacctgaagcctttcgagcgggacatcagcaccgagatctaccaggctggcagcaccccttgtaacggcgtggaaggcttcaactgctacttcccactgcagtcctacggctttcagcctaccaatggcgtgggctatcagccctacagagtggtggtgctgtccttcgagctgctgcatgctcctgctaccgtgtgcggccctaagaaatctaccaacctggtcaagaacaaatgcgtgaacttcaacttcaacggcctgaccggcaccggcgtgctgacagagtccaacaagaagttcctgccattccagcagttcggccgggatatcgccgataccacagatgccgtcagggaccctcagacactggaaatcctggacatcaccccttgctccttcggcggagtgtctgtgatcaccccaggcaccaacacctctaaccaggtggccgtgctgtatcaggacgtgaactgtaccgaggtgcccgtggctatccatgccgatcagctgacccctacatggcgcgtgtactccaccggctctaacgtgttccagacaagagctggctgtctgatcggcgctgagcacgtgaacaattcctacgagtgcgacatccccatcggagccggaatctgcgcctcttatcagacccagaccaactctcccagacgggccagatctgtggccagccagtctatcattgcttacaccatgagcctgggcgccgagaactctgtggcctacagcaacaactctatcgctatccccaccaacttcaccatctccgtgaccacagagatcctgccagtgtccatgaccaagaccagcgtggactgcaccatgtacatctgcggcgactctaccgagtgctccaacctgctgctccagtacggctccttctgcacccagctgaatagagccctgaccggaatcgccgtggaacaggacaagaacacccaagaggtgttcgcccaagtgaagcagatctacaagacccctcctatcaaggacttcggcggcttcaatttctcccagattctgcccgatcctagcaagccctccaagcggtctttcatcgaggacctgctgttcaacaaagtgacactggccgacgccggcttcatcaagcagtacggcgactgtctgggcgacattgccgctagggatctgatctgcgcccagaagtttaacggactgacagtgctgcctcctctgctgaccgatgagatgatcgcccagtacacctccgcactgctggctggcacaatcacctctggatggacatttggcgctggcgctgctctgcaaatcccattcgctatgcaaatggcctaccggttcaacggcatcggcgtgacccagaatgtgctgtacgagaaccagaagctgatcgccaaccagttcaacagcgccatcggaaagatccaggacagcctgtccagcaccgcttctgccctgggaaagctgcaggatgtggtcaaccagaacgctcaggccctgaacaccctcgtgaagcagctgtctagcaacttcggcgccatctcctctgtgctgaacgatatcctgagccggctggacaaggtggaagccgaggtgcagatcgacagactgatcaccggacggctgcagtccctgcagacctatgttacccagcagctgatccgggctgccgagattagagcctctgccaatctggccgcaaccaagatgtctgagtgtgtgctgggacagtccaagagagtggacttctgcggcaagggctaccacctgatgagcttccctcagtctgctcctcacggcgtggtgtttctgcacgtgacctacgtgcccgctcaagagaagaactttaccaccgctcctgccatctgccacgacggcaaggctcactttcctagagaaggcgtgttcgtgtctaacggcacccattggttcgtgacacagcggaacttctacgagccccagatcatcaccaccgacaacaccttcgtgtccggcaactgcgacgtcgtgatcggaattgtgaacaataccgtgtacgaccctctgcagcccgagctggactccttcaaagaggaactggacaagtactttaagaaccacacaagccccgacgtggacctgggagacatctctggcatcaacgcctccgtggtcaacatccagaaagagatcgaccggctgaacgaggtggccaagaatctgaacgagtccctgatcgacctgcaagaactggggaagtacgagcagtacatcaagtggccctggtacatctggctgggctttatcgctggcctgatcgctatcgtgatggtcacaatcatgctgtgctgtatgacctcctgttgctcctgcctgaagggctgctgctcttgcggctcttgctgcaagttcgacgaggacgactctgagcccgtgctgaaaggcgtgaagctgcactatacctgatgactcgag

SEQ ID NO: 33 - protein, 1513 aa, Artificial Sequence
  HBSAg-(EAAAK)3-full-length SARS-CoV-2 spike protein
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIEAAAKEAAAKEAAAKFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2024/0108715 A1
(43) Pub. Date: Apr. 4, 2024

(54) 2019-NCOV (SARS-COV-2) VACCINE

(71) Applicant: VAXBIO LTD, Oxfordshire (GB)

(72) Inventors: Gaurav GUPTA, Oxfordshire (GB); Reinhard GLUECK, Caltagirone (IT)

(21) Appl. No.: 17/800,472

(22) PCT Filed: Feb. 17, 2021
(86) PCT No.: PCT/GB2021/050383

§ 371 Date: Aug. 17, 2022

(30) Foreign Application Priority Data
Feb. 17, 2020 (GB) 2002166.3

(51) Int. Cl. A61K 39/215; A61P 31/14; C07K 14/005; C12N 7/00

(52) U.S. Cl. CPC A61K 39/215; A61P 31/14; C07K 14/005; C12N 7/00; A61K 2039/5258

(57) ABSTRACT

The present invention relates to Coronavirus 2019-nCOV spike protein, polynucleotides encoding said spike protein, antibodies and vaccines for treatment or prevention of 2019-nCOV infection.

### RELATED APPLICATIONS

This application is a national phase application claiming benefit of priority under 35 U.S.C. § 371 to Patent Convention Treaty (PCT) International Application serial number PCT/GB2021/050383, filed Feb. 17, 2021, now pending, which claims the benefit of priority to GB 2002166.3, filed Feb. 17, 2020. The aforementioned applications are expressly incorporated herein by reference in their entirety and for all purposes.

### FIELD OF THE INVENTION

The present invention relates to Coronavirus 2019-nCOV spike protein, polynucleotides encoding said spike protein, antibodies and vaccines for treatment or prevention of 2019-nCOV infection.

### BACKGROUND OF THE INVENTION

Since 8 Dec. 2019, several cases of pneumonia of unknown aetiology have been reported in Wuhan, Hubei province, China. Most patients worked at or lived around the local Huanan seafood wholesale market, where live animals were also on sale. In the early stages of this pneumonia, severe acute respiratory infection symptoms occurred, with some patients rapidly developing acute respiratory distress syndrome (ARDS), acute respiratory failure, and other serious complications. On 7 Jan. 2020, a novel coronavirus was identified by the Chinese Center for Disease Control and Prevention (CDC) from the throat swab sample of a patient, and was subsequently named 2019-nCOV by WHO and has now been designated SARS-COV-2.

Coronaviruses can cause multiple system infections in various animals and mainly respiratory tract infections in humans, such as severe acute respiratory syndrome (SARS) and Middle East respiratory syndrome (MERS). Most patients have mild symptoms and good prognosis.

So far, a few patients with 2019-nCOV have developed severe pneumonia, pulmonary oedema, ARDS, or multiple organ failure and have died. All costs of 2019-nCOV treatment are covered by medical insurance in China. At present, information regarding the epidemiology and clinical features of pneumonia caused by 2019-nCOV is scarce and no vaccine is available. Therefore, there is an ongoing need for the development of antigens which may be used in vaccines to prevent and treat 2019-nCoV infection. Further, there is a need to provide antigens that can be produced at scale inexpensively.

The present invention addresses one or more of the above needs by providing polynucleotides encoding 2019-nCOV antigens, particularly antigens from the spike protein of 2019-nCOV, vectors comprising said polynucleotide, vectors encoding said antigens, and binding compounds (particularly antibodies and antibody-like molecules including aptamers and peptides) raised against the antigen, together with the use thereof (either alone or in combination) in the prevention or treatment of infection with 2019-nCOV. The polynucleotides encoding the antigen are optimised for expression in host cells of interest. Antibodies and antibody-like molecules raised against the antigen may bind (e.g. specifically bind) to the antigen.

### SUMMARY OF THE INVENTION

To-date, no vaccine has been developed for 2019-nCOV. The present inventors have developed polynucleotides encoding the 2019-nCOV spike protein, said polynucleotides being optimised for expression in commonly used expression systems. These polynucleotides provide increased level and duration of expression of the 2019-nCOV spike protein, making them advantageous for large-scale production of this antigen. Furthermore, the polynucleotides devised by the inventors encode the spike protein amino acid sequence in a form which retains the conformation of the native spike protein. Thus, the spike protein produced according to the present invention can give rise to an immunoprotective response, particularly through the production of neutralising antibodies.

Accordingly, the present invention provides an isolated polynucleotide encoding a spike protein from 2019-nCOV having at least 90% identity with SEQ ID NO: 1, or a fragment thereof that has a common antigenic cross-reactivity with said spike protein, wherein said polynucleotide is optimised for recombinant expression.

Said polynucleotide of may be optimised for expression in a host cell selected from: Escherichia coli; yeast, preferably Komagataella or Saccharomyces; and/or mammalian cells, preferably human cells. Optimisation may occur by omitting one or more cis-acting sequence motif, said one or more cis-acting sequence motif being independently selected from: an internal TATA-box; a chi-site; a ribosomal entry site; an AT-rich and/or GC-rich stretch of sequence; an RNA instability motif; a repeat sequence and/or an RNA secondary structure; a cryptic splice donor site; a cryptic splice acceptance site; and/or any combination of (a) to (i). Said polynucleotide may integrate into the host cell genome. Said polynucleotide may have a codon adaptation index (CAI) of at least about 0.80, preferably at least about 0.9, more preferably at least about 0.93. A polynucleotide of the invention may comprise or consist of a nucleic acid sequence having at least 90% identity to any one of SEQ ID NO: 2 to 8, 13, 14, 26, 27, 28, 30 or 32. The polynucleotide of the invention typically encodes a spike protein, or fragment thereof which: (i) retains the conformational epitopes present in the native 2019-nCOV spike protein; (ii) results in the production of neutralising antibodies specific for the spike protein or fragment thereof when the nucleic acid or the encoded spike protein or fragment thereof is administered to a subject; and/or (iii) comprises or consists of receptor-binding domain (RBD) of the 2019-nCOV spike protein, preferably having at least 90% identity with SEQ ID NO: 15.

The invention further provides an expression construct comprising polynucleotide of the invention, operably linked to a promoter.

The invention further provides a vaccine composition comprising a spike protein from 2019-nCOV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, preferably having at least 90% identity with SEQ ID NO: 15. Said vaccine typically results in the production of neutralising antibodies specific for the spike protein or fragment thereof when administered to a subject.

The invention also provides a viral vector, RNA vaccine or DNA plasmid that expresses a spike protein from 2019-nCOV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, preferably having at least 90% identity with SEQ ID NO: 15. Said viral vector, RNA vaccine or DNA plasmid may further encode a signal peptide. The signal peptide may direct secretion from human cells. The viral vector, RNA vaccine or DNA plasmid of the invention may further express one or more additional antigen or a fragment thereof, preferably one or more additional antigen from 2019-nCOV, or a fragment thereof. The spike protein or fragment thereof and the one or more additional antigen or fragment thereof may be expressed: as a fusion protein; or in separate viral vectors, RNA vaccines or DNA plasmids for use in combination. Said viral vector, RNA vaccine or DNA plasmid may comprise one or more polynucleotide or expression construct of the invention.

The invention also provides a fusion protein comprising a spike protein from 2019-nCOV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, preferably having at least 90% identity with SEQ ID NO: 15. Said VLP or fusion protein may further comprise: the Hepatitis B surface antigen (HBSAg), or a fragment thereof that has a common antigenic cross-reactivity with said HBSAg; the HPV 18 L1 protein, or a fragment thereof that has a common antigenic cross-reactivity with said HPV 18 L1 protein; the Hepatitis E P239 protein (HEV), or a fragment thereof that has a common antigenic cross-reactivity with said Hepatitis E P239 protein; and/or the HPV 16 L1 protein, or a fragment thereof that has a common antigenic cross-reactivity with said HPV 16 L1 protein. The fusion protein may be encoded by a polynucleotide which comprises or consists of a nucleic acid sequence having at least 90% identity with any one of SEQ ID NO: 3, 5, 6, 8, 26, 27, 29,30, or 32; and/or may the fusion protein may comprise of consist of an amino acid sequence having at least 90% identity with any one of SEQ ID NO: 9, 10, 11, 12, 28,31, or 33.

The invention also provides a virus-like particle comprising a spike protein from 2019-nCoV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, preferably having at least 90% identity with SEQ ID NO: 15. Preferably the VLP comprises or consists of a fusion protein of the invention.

The invention also provides an antibody, or binding fragment thereof, that specifically binds to a 2091-nCOV spike protein antigen, or fragment thereof, as herein. Said antibody, or binding fragment thereof, may be a monoclonal or polyclonal antibody. Said antibody, or binding fragment thereof may be an Fab, F(ab′)2, Fv, scFv, Fd or dAb.

The invention further provides an oligonucleotide aptamer that specifically binds to a 2019-nCOV spike protein or fragment thereof as defined herein.

The invention provides a vaccine composition comprising the viral vector, and/or RNA vaccine and/or DNA plasmid of the invention.

The invention also provides a polynucleotide of the invention, and/or the expression construct of the invention, and/or vaccine composition of the invention, and/or the viral vector and/or RNA vaccine and/or DNA plasmid of the invention and/or the virus-like particle of the invention, and/or the fusion protein of the invention, and/or the antibody of the invention and/or the aptamer of the invention for use in the treatment and/or prevention of 2019-nCoV infection.

The invention also provides the use of a polynucleotide of the invention, and/or the expression construct of the invention, and/or vaccine composition of the invention, and/or the viral vector and/or RNA vaccine and/or DNA plasmid of the invention and/or the virus-like particle of the invention, and/or the fusion protein of the invention, and/or the antibody of the invention and/or the aptamer of the invention in the manufacture of a medicament for the prevention and/or treatment of 2019-nCoV infection.

The invention also provides a method of producing a spike protein from 2019-nCOV having at least 90% identity with SEQ ID NO: 1, or a fragment thereof, comprising expressing a polynucleotide of the invention in a host cell, and optionally purifying the spike protein or fragment. Said method may further comprise formulating said spike protein or fragment thereof with a pharmaceutically acceptable carrier or diluent.

### DESCRIPTION OF FIGURES

FIG. 1: Schematic of the coronavirus's structure and the function of the structural proteins.

FIG. 2: Tabulated results from ELISA reporting antibody titre at day0 and day 14 following administration of 2019-nCOV spike protein and fusion proteins comprising 2019-nCOV spike protein produced according to the invention.

FIG. 3: Western blots showing secreted protein HBSAg(EAAAK)₃RBD released into culture medium after 40 h (following concentration by centrifugal filtration). Left blot=HBSAg antibody. Right blot=2019-nCOV antibody. For both blots, lane M=marker; lane 1=HEV-GGGS-RBD #10B; lane 2=HBSAg-EAAK-RBD #2A (293F secreted, total protein content by Bradford assay=750 μg/ml); lane 3=HBSAg-EAAAK-RBD #2B (293F secreted, total protein content by Bradford assay=500 μg/ml); lane 4=CoV-s #17 (293F surface bounded HisTag) FIG. 4: Graphs illustrating titres at d14 (A) and d42 (B) in mice inoculated with HBSAg(EAAAK)₃RBD generated in HEK cells, either alone, or using aluminium hydroxide or Addavax™ adjuvants.

FIG. 5: Western blots showing secreted protein HEV-(GGGGS)₃-RBD produced by E. coli. Left blot=#10A. Middle blot=#10B. Right blot=#10C. For all blots an anti-HEV mAB was used at a dilution of 1:4000.

FIG. 6: Graphs illustrating titres at d14 (A) and d42 (B) in mice inoculated with HEV-GGGGS-RBD generated in E. coli, either alone, or using aluminium hydroxide or Addavax™ adjuvants.

FIG. 7: Western blot of recombinant HBSAg-(EAAAK)₃-full-length 2019-nCOV spike protein fusion protein (HBSAg-(EAAAK)₃-CoV-s) clone D8-SA01-01-01 (4×) and clone D8-SA01-02-01 (5X) expressed in HEK 293 cells.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The publications discussed herein are provided solely for their disclosure prior to the filing date of the present application. Nothing herein is to be construed as an admission that such publications constitute prior art to the claims appended hereto.

### Coronaviruses

Coronaviruses (CoVs) belong to the subfamily Coronavirinae, in the family Coronaviridae of the order Nidovirales. There are four genera:Alphacoronavirus, Betacoronavirus, Gammacoronavirus and Deltacoronavirus. Alphacoronaviruses and Betacoronaviruses infect species of mammal, Gammacoronaviruses infect species of bird, and Deltacoronaviruses infect both species of mammals and birds.

CoVs are large enveloped single positive-sense RNA viruses. Mutation rates of RNA viruses are greater than DNA viruses, suggesting a more efficient adaptation process for survival.

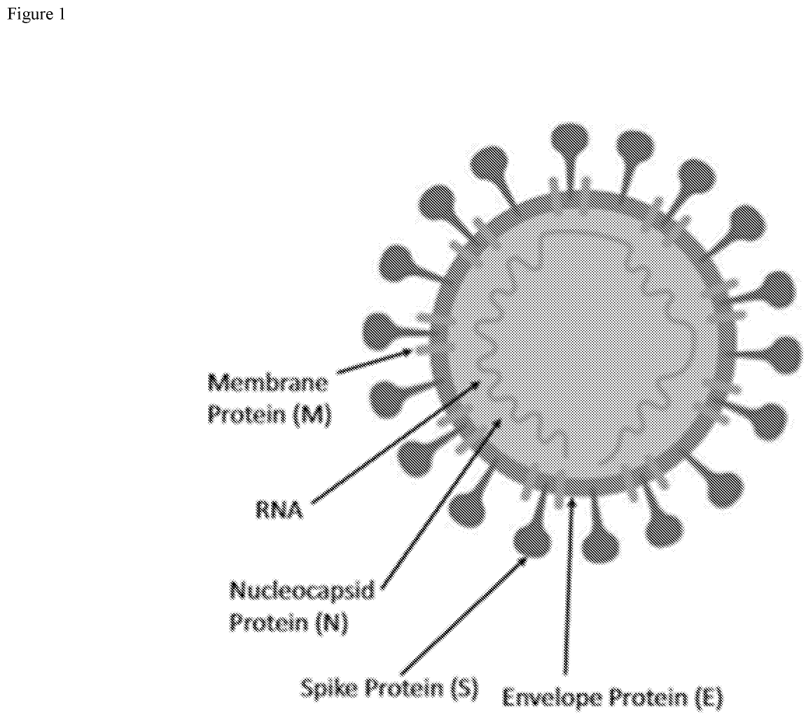

CoVs have the largest genome among all RNA viruses, typically ranging from 27 to 32 kb. The CoV genome codes for at least four main structural proteins: spike (S), membrane (M), envelope (E), nucleocapsid (N) proteins and other accessory proteins which aid the replicative processes and facilitate entry into cells. FIG. 1 summarises the coronavirus's structure and the function of the structural proteins. Briefly, the CoV genome is packed inside a helical capsid formed by the nucleocapsid and further surrounded by an envelope. Associated with the viral envelope are at least three structural proteins: the membrane and envelope proteins, which are involved in virus assembly, and the spike protein, which mediates virus entry into host cells. Some coronaviruses also encode an envelope-associated hemagglutinin-esterase protein (HE). The spike protein forms large protrusions from the virus surface, giving coronaviruses the appearance of having crowns, from which the name “Coronavirus” is derived. As well as mediating virus entry, the spike protein is a critical determinant of viral host range and tissue tropism and a major inducer of host immune responses.

2019-nCOV (officially named severe acute respiratory syndrome coronavirus 2, SARS-CoV-2, the two terms being used interchangeably herein) is the causative agent of coronavirus disease 2019 (COVID-19) and is contagious among humans. It is believed that 2019-nCOV originated in animals, with bats being a likely source given the genetic similarities of 2019-nCOV to SARS-COV (79.5%) and bat coronaviruses (96%). Any disclosure herein in relation to CoVs also applies directly and without restriction to 2019-nCOV.

The CoV spike protein comprises three domains: (i) a large ectodomain; (ii) a transmembrane domain (which passes through the viral envelope in a single pass); and (iii) a short intracellular tail. The ectodomain consists of three receptor-binding subunits (3×S1) and a trimeric stalk made of three membrane-fusion subunits (3×S2). During virus entry, SI binds to a receptor on the host cell surface for viral attachment, and S2 fuses the host and viral membranes, allowing viral genomes to enter host cells. Receptor binding and membrane fusion are the initial and critical steps in the coronavirus infection cycle. There is significant divergence in the receptors targeted by different CoVs.

The 2019-nCOV spike protein or immunogenic fragments thereof have therapeutic potential as antigens for vaccines against 2019-nCoV infection.

Accordingly, as described herein, the invention relates to a 2019-nCOV spike protein has at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably the invention relates to a spike protein from 2019-nCOV has at least 90%, at least 95%, at least 96%, at least 97%, at least 98%, at least 99% or more identity with SEQ ID NO: 1, or a fragment thereof, that has a common antigenic cross-reactivity with said spike protein. More preferably, the invention relates to a spike protein from 2019-nCOV having least 98%, at least 99% or more with SEQ ID NO: 1, or a fragment thereof, that has a common antigenic cross-reactivity with said spike protein. The spike protein from 2019-nCOV may comprise or consist of SEQ ID NO: 1, or a fragment thereof, that has a common antigenic cross-reactivity with said spike protein.

According to the present invention, the 2019-nCOV spike protein or fragment thereof encoded by a polynucleotide of the invention maintains one or more conformational epitope present in native 2019-nCOV spike protein. As such, the 2019-nCOV spike protein or fragment thereof encoded by a polynucleotide of the invention is capable of giving rise to an immunoprotective effect. Typically said immunoprotective effect comprises the production of neutralising antibodies (nAb) which specifically bind to the one or more conformational epitope of the 2019-nCOV spike protein or fragment thereof encoded by a polynucleotide of the invention. A conformational epitope of a CoV spike protein has a specific three-dimensional structure that is found in the tertiary structure of the CoV spike protein. Said one or more conformational epitope is typically within the ectodomain of the spike protein. Preferably the 2019-nCOV spike protein or fragment thereof encoded by a polynucleotide of the invention retains all of the conformational epitopes present in native 2019-nCOV spike protein.

In some preferred embodiments, the invention relates to an immunogenic fragment of 2019-nCoV spike protein which is the receptor-binding domain (RBD) of the 2019-nCOV spike protein. This RBD is responsible for 2019-nCOV binding to a host cell and thus facilitates entry of 2019-nCOV particles into the host cell. The RBD corresponds to amino acid residues 319 to 529 of SEQ ID NO: 1, as described herein is referred to as SEQ ID NO: 15. The RBD is encoded by bases corresponding to positions 955 to 1597 in the genome of the2019-nCOV virus (Genbank Accession No. MN908947, version 3 of which (MN908947.3) was deposited 17 Jan. 2020). Accordingly, as described herein, the invention relates to an RBD of the 2019-nCOV spike protein has at least 70%, at least 75%, at least 80%, at least 90%, at least 95%, at least 96%, at least 97%, at least 98%, at least 99% or more identity with SEQ ID NO: 15. Preferably the invention relates to an RBD of the 2019-nCOV spike protein that has at least 90%, at least 95%, at least 96%, at least 97%, at least 98%, at least 99% or more identity with SEQ ID NO: 15. More preferably, the invention relates to an RBD of the 2019-nCOV spike protein having least 98%, at least 99% or more with SEQ ID NO: 15. The RBD of the 2019-nCOV spike protein may comprise or consist of SEQ ID NO: 15. Any and all disclosure herein relating to the 2019-nCOV spike protein (e.g. in relation to polynucleotides, viral vectors, DNA plasmids, RNA vaccines, virus-like particles (VLPs), fusion proteins, antibodies, compositions and pharmaceutical compositions, formulations and therapeutic indications) applies equally and without reservation to the RBD of the 2019-nCOV spike protein. References herein to RBD refer to the RBD of the 2019-nCOV spike protein.

CoVs are large enveloped single positive-sense RNA viruses. Mutation rates of RNA viruses are greater than DNA viruses, suggesting a more efficient adaptation process for survival. Thus, there is a risk that antigenic drift will also become a feature of the 2019-nCOV, or if 2019-nCOV becomes endemic in the population once the pandemic has subsided. Indeed, research to-date has already identified mutations within the receptor binding domain (RBD) of the spike protein of 2019-nCOV, particularly G476S and V483A/G, as well as a prevalent D614G mutation in the vicinity of the S1/S2 site (Saha et al., ChemRxiv™ http://doi.org/10.26434/chemrxiv.12320567.v1), which the evidence suggests can enhance cell entry by the 2019-nCOV virion, and also broaden the host cell tropism. Other mutations reported in the 2019-nCOV spike protein include S943 (particularly S943P), L5 (particularly L5F), L8 (particularly L8F), V367 (particularly V367F), H49 (particularly H49Y), Y145 (particularly Y145H/del), Q239 (particularly Q239K), A831 (particularly A831V), D839 (particularly D839Y/N/E), and P1263 (particularly P1263L), or any combination thereof (Korber et al., BioRxiv™ https://doi.org/10.1101/2020.04.29.069054).

Accordingly, the invention advantageously allow 2019-nCOV vaccine antigens to be modified if required to provide enhanced immunity against strains with mutated spike proteins as they arise. By way of non-limiting example, any 2019-nCOV spike protein or fragment thereof according to the invention may be modified (particularly by substitution) at position (i) D614, (ii) V483, (iii) G476, (iv) K417, (v), E484, (vi) N501, (vii) A570, and (viii) P681, or any combination of (including any two, any three, any four, any five, any six, any seven or all eight) of (i) to (viii). Alternatively or in addition, the 2019-nCOV spike proteins or fragments thereof may comprise deletion mutations, including deletions at one or more of amino acid residues 69, 70 and/or 144. As described herein, the positions of the mutations/modifications typically corresponds to the numbering of amino acids in SEQ ID NO: 1 of the present invention.

Modification at position D614, particularly the D614G substitution, is preferred. In particular, any 2019-nCOV spike protein or fragment thereof according to the invention may comprise the following substitutions (i) G476S, (ii) V483A/G, (iii) D614G, (iv) K417N/T, (v), E484K, (vi) N501Y, (vii) A570D, and (viii) P681H, or any combination of (including any two, any three, any four, any five, any six, any seven or all eight) of (i) to (viii).

The invention also relates to 2019-nCOV spike proteins or fragments thereof from a variant 2019-nCOV. In particular, the invention may relate to 2019-nCOV spike proteins or fragments thereof from the B.1.1.7 strain (also known as 201/501Y.V1, which was first detected in the UK); the B.1.351 strain (also known as 20H/501. V2, which was first detected in South Africa), and/or the Pl strain (also known as 20J/501Y.V3, which was first detected in Japan and Brazil). The key mutations of the B.1.1.7 stain comprise deletion of residues 69/70 and 144Y, as well as N501Y, A570D, D614G and P681H substitutions. The key mutations of the B.1.351 strain comprise K417N, E484K, N501Y and D614G substitutions. The key mutations of the P.1 strain comprise E484K, K417N.T, N501Y and D614G.

All the disclosure herein in relation to polynucleotides, spike proteins and fragments thereof, VLPs, fusion proteins and DNA/RNA vaccine applies equally to different variants and strains of 2019-nCOV unless explicitly stated.

### Polynucleotides

The present invention provides a polynucleotide that encodes or expresses (the terms “encode” and “express” are used interchangeably herein) the protein or immunogenic fragment of the invention. The term polynucleotide encompasses both DNA and RNA sequences. Herein, the terms “nucleic acid”, “nucleic acid molecule” and “polynucleotide” are used interchangeably.

The invention provides an isolated polynucleotide encoding a spike protein from 2019-nCoV having at least 90% identity with SEQ ID NO: 1, or a fragment thereof that has a common antigenic cross-reactivity with said spike protein. For example, the polynucleotide may encode an RBD of the 2019-nCOV spike protein, preferably wherein said RBD has at least 90% identity with SEQ ID NO: 15. Exemplary polynucleotides encoding the RBD are shown in SEQ ID NO: 13, and the codon-optimised sequence of SEQ ID NO: 14.

The invention also encompasses polynucleotides encoding a variant spike protein from 2019-nCOV, as described above, or fragments thereof that have common antigenic cross-reactivity with said variant spike protein. Said varint spike proteins typically have at least 90% identity with SEQ ID NO: 1.

A polynucleotide of the invention may be used for recombinant expression of the protein or immunogenic fragment of the invention (including in the form of a VLP or fusion protein), or as a DNA/RNA vaccine.

The present inventors are the first to provide improved polynucleotides encoding the 2019-nCOV spike protein or immunogenic fragments thereof. In particular, the present inventors have designed polynucleotides that are optimised for recombinant expression. A polynucleotide of the invention may be optimised for expression in one or more particular cell type, for example, eukaryotic cells (e.g. mammalian cells, yeast cells, insect cells or plants cells) or prokaryotic cells (bacterial cells). Typically the polynucleotides are optimised for expression in bacterial cells, yeast cells or mammalian cells. Preferably said polynucleotides are optimised for expression in Escherichia coli (for example, BL21(DE3), RV308(DE3), HMS174(DE3) or K12 strains), Komagataella (formally assigned as Pichia, particularly Komagataella pastoris or Komagataella phaffii), Saccharomyces (particularly Saccharomyces cerevisiae) or human cells (preferably 293 F cells, HEK 293 cells, HEK 293T cells or Hela cells). Other cell types/expression systems of interest include Pichia angusta, Hansenula polymorpha, Chinese Hamster Ovary (CHO) cells and/or insect cell baculovirus-based expression systems.

The term “optimised” as used herein relates to optimisation for recombinant expression of the 2019-nCOV spike protein or immunogenic fragment thereof, and includes both codon optimisation and/or other modifications to the polynucleotide (both in terms of the nucleic acid sequence and other modifications) which increase the level and/or duration of expression of the 2019-nCOV spike protein from the polynucleotide within the host cell/organism, or which otherwise provide an advantage when expressing the 2019-nCOV spike protein, or fragment thereof, from a polynucleotide of the invention.

The term “codon optimised” refers to the replacement of at least one codon within a base polynucleotide sequence with a codon that is preferentially used by the host organism or cell in which the polynucleotide is to be expressed. Typically, the most frequently used codons in the host organism are used in the codon-optimised polynucleotide sequence. Methods of codon optimisation are well known in the art.

By way of non-limiting example, another form of polynucleotide optimisation are modifications which minimise RNA structure, as structures that involve or otherwise occlude the RBS and/or start codon in genes expressed in prokaryotes can impair expression. Optimisation also encompasses modifications to the polynucleotide which optimise translation, either by increasing the rate of translation, or by balancing the rate of translation with the need to allow for efficient “self” or chaperone-aided protein folding, in which strategically placed slower codons or codon runs (e.g. at protein domain boundaries) could maximise folding efficiency whilst maintaining a high overall translation rate. Optimisation may also encompass the removal of deleterious motifs within the nucleic acid sequence of the polynucleotide. By way of non-limiting example, expressing a gene under control of a T7 promoter in E. coli, it is preferable to avoid both class I and II transcriptional termination sites. Shine-Dalgarno-like sequences within the coding sequence may cause incorrect downstream initiation or translational pauses in prokaryotic hosts. For expression in eukaryotic hosts/cells, potential splice signals, polyadenylation signals and other motifs affecting mRNA processing and stability may be removed. Other classes of deleterious motifs include sequences that promote ribosomal frameshifts and pauses. Any combination of modifications may be made to the polynucleotides of the invention to optimise expression in a host cell of interest.

Typically polynucleotides of the invention optimised for expression in bacterial cells, particularly E. coli, include cloned N-terminal and/or C-terminal deleted amino acids. Preferably about 1 to 20, more preferably about 1 to 15, most preferably about 5 to 10 cloned N-terminal and/or C-terminal deleted amino acids are included.

It will be understood by a skilled person that numerous different polynucleotides can encode the same polypeptide as a result of the degeneracy of the genetic code. It is also understood that skilled persons may, using routine techniques, make nucleotide substitutions that do not affect the polypeptide sequence encoded by the nucleic acid molecules to reflect the codon usage of any particular host organism in which the polypeptides are to be expressed. Therefore, unless otherwise specified, a “polynucleotide that encodes the protein or immunogenic fragment of the invention” includes all polynucleotide sequences that are degenerate versions of each other and that encode the same amino acid sequence.

A polynucleotide of the invention is typically designed so that it is capable of integrating into the genome of a host cell of interest. Different optimisation strategies may be used to facilitate integration depending on the desired host cell.

Typically a polynucleotide of the invention is optimised by the removal or omission of one or more cis-acting sequence motif (also referred to interchangeably as cis-acting elements or cis-acting regulatory elements). A cis-acting sequence motif is a sequence in the vicinity of the structural portion of a gene that is required for gene expression. Said one or more cis-acting sequence motif may be independently selected from: (a) an internal TATA-box; (b) a Chi-site; (c) a ribosomal entry site; (d) an AT-rich and/or GC-rich stretch of sequence; (e) an RNA instability motif; (f) a repeat sequence and/or an RNA secondary structure; (g) a cryptic splice donor site; (h) a cryptic splice acceptance site; and/or (i) any combination of (a) to (i). These cis-acting sequence motifs are known in the art. By way of non-limiting example, regions of high GC content (e.g.

above about 70%, preferably above about 80%) and/or low GB content (e.g. below about 40%, preferably below about 30%) are omitted. Preferably both regions of high and low GC content are omitted, in combination with the removal or omission of one or more other cis-acting sequence motif.

A polynucleotide of the invention may also be “codon optimised” as described herein. Codon optimisation preferably occurs in addition to the removal or omission of one or more cis-acting sequence motif as described herein.

The average GC content of a polynucleotide of the invention may also be modified to optimise expression of said polynucleotide. For example, the average GC content of a polynucleotide may be in the region of about 40% to about 60%, preferably about 40% to about 57%, more preferably about 45% to about 56%.

A polynucleotide of the invention typically has a codon adaptation index (CAI) of at least about 0.80, preferably at least about 0.9, more preferably at least about 0.91, at least about 0.92, at least about 0.93, at least about 0.94, at least about 0.95, at least about 0.96, at least about 0.97, at least about 0.98, at least about 0.99, or more up to about 1.0.

As a result of the optimising modifications, a polynucleotide of the invention may increase the expression of the encoded 2019-nCOVspike protein or fragment thereof by at least 20%, at least 30%, at least 40%, at least 50%, at least 60%, at least 70%, at least 80%, at least 90%, at least 100% or more compared with the corresponding non-optimised polynucleotide sequence. Preferably the expression level is increased by at least 50%, at least 60%, at least 70%, at least 80%, at least 90%, at least 100% or more, more preferably at least at least 70%, at least 80%, at least 90%, at least 100% or more compared with the corresponding non-optimised polynucleotide.

A polynucleotide of the invention may be capable of expression in the host cell for at least one week, at least two weeks, at least three weeks, at least one month, at least two months, at least three months, at least four months or more, preferably at least one month, at least two months, at least three months, at least four months or more.

The inventors have demonstrated that 2019-nCOV spike protein and fusion proteins comprising 2019-nCOV spike protein can be expressed at high levels in a variety of expression systems/host cells using their rationally designed optimised polynucleotides. Furthermore, the present inventions have surprisingly demonstrated that 2019-nCOV spike protein and fusion proteins comprising 2019-nCOV spike protein can generate a strong antibody response in mice, which demonstrates their potential therapeutic utility. The inventors have exemplified their optimisation methodology by designing and generating optimised polynucleotides and fusion proteins as described in the Examples below.

Accordingly, a polynucleotide of the invention may comprise or consist of a nucleic acid sequence having at least 70%, at least 75%, at least 80%, at least 90%, at least 95%, at least 96%, at least 97%, at least 98%, at least 99% or more identity to any one of SEQ ID NOs: 2, 3, 4, 5, 6, 7 or 8. Preferably a polynucleotide of the invention may comprise or consist of a nucleic acid sequence having at least 90%, at least 95%, at least 96%, at least 97%, at least 98%, at least 99% or more identity to any one of SEQ ID NOs: 2, 3, 4, 5, 6, 7, 8, 13, 14, 26, 27, 29,30, or 32. More preferably, a polynucleotide of the invention may comprise or consist of a nucleic acid sequence having at least 98%, at least 99% or more identity to any one of SEQ ID NOs: 2, 3, 4, 5, 6, 7, 8, 13, 14, 26, 27, 29,30, or 32. A polynucleotide of the invention may comprise or consist of the nucleic acid sequence of any one of SEQ ID NOs: 2, 3, 4, 5, 6, 7, 8, 13, 14, 26, 27, 29, 30, or 32. In addition, the 5′ cloning site, the 3′ cloning site, or the 5′ and 3′ cloning sites identified in any of SEQ ID NOs; 2, 3, 4, 5, 6, 7, 8, 13, 14, 26, 27, 29, 30, or 32, or any variant thereof as described herein, may be deleted. Thus, the invention provides polynucleotides comprising or consisting of any one of SEQ ID NOs: 2, 3, 4, 5, 6, 7, 8, 13, 14, 26, 27, 29,30, or 32 but lacking the 5′ cloning site, the 3′ cloning site, or the 5′ and 3′ cloning sites identified in any of SEQ ID NOs; 2, 3, 4, 5, 6, 7, 8, 13, 14, 26, 27, 29,30, or 32. Alternatively, the 5′ cloning site, the 3′ cloning site, or the 5′ and 3′ cloning sites identified in any of SEQ ID NOs; 2, 3, 4, 5, 6, 7, 8, 13, 14, 26, 27, 29, 30, or 32, or any variant thereof as described herein, may be independently replaced with another appropriate cloning site. Suitable alternative cloning sites are well known in the art.

The invention particularly provides polynucleotides encoding an RBD of the 2019-nCOV spike protein. Accordingly, a polynucleotide of the invention may comprise or consist of a nucleic acid sequence having at least 70%, at least 75%, at least 80%, at least 90%, at least 95%, at least 96%, at least 97%, at least 98%, at least 99% or more identity to SEQ ID NO: 13, or to the codon-optimised sequence of SEQ ID NO: 14. Preferably a polynucleotide of the invention may comprise or consist of a nucleic acid sequence having at least 90%, at least 95%, at least 96%, at least 97%, at least 98%, at least 99% or more identity to SEQ ID NO: 13, or to the codon-optimised sequence of SEQ ID NO: 14. More preferably, a polynucleotide of the invention may comprise or consist of a nucleic acid sequence having at least 98%, at least 99% or more identity to SEQ ID NO: 13, or to the codon-optimised sequence of SEQ ID NO: 14. A polynucleotide of the invention may comprise or consist of the nucleic acid sequence of SEQ ID NO: 13, or the codon-optimised sequence of SEQ ID NO: 14.

A polynucleotide of the invention typically encodes a 2019-nCOV spike protein, or an immunogenic fragment thereof which: (a) retains the conformational epitopes present in the native 2019-nCOV spike protein; and/or (b) results in the production of neutralising antibodies specific for the spike protein or fragment thereof when the nucleic acid or the encoded spike protein or fragment thereof is administered to a subject.

The polynucleotide of the invention typically expresses a spike protein from 2019-nCOV having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably a polynucleotide of the invention expresses a spike protein from 2019-nCOV having at least 90%, at least 95%, at least 96%, at least 97%, at least 98%, at least 99% or more identity with SEQ ID NO: 1, or a fragment thereof, that has a common antigenic cross-reactivity with said spike protein. More preferably, a polynucleotide of the invention expresses a spike protein from 2019-nCOV having least 98%, at least 99% or more with SEQ ID NO: 1, or a fragment thereof, that has a common antigenic cross-reactivity with said spike protein. A polynucleotide of the invention may express a spike protein from 2019-nCOV comprising or consisting of SEQ ID NO: 1, or a fragment thereof, that has a common antigenic cross-reactivity with said spike protein.

A polynucleotide of the invention may be comprised in an expression construct to facilitate expression of the 2019-nCOV spike protein or fragment thereof. Accordingly, the invention further provides an expression construct comprising polynucleotide the invention. Typically, in such an expression construct a polynucleotide of the invention is operably linked to a suitable promoter. The polynucleotide may be linked to a suitable terminator sequence. The polynucleotide may be linked to both a promoter and terminator. Suitable promoter and terminator sequences are well known in the art.

The choice of promoter will depend on where the ultimate expression of the polynucleotide will take place. In general, constitutive promoters are preferred, but inducible promoters may likewise be used. The construct produced in this manner includes at least one part of a vector, in particular regulatory elements. The vector is preferably capable of expressing the nucleic acid in a given host cell. Any appropriate host cell may be used, such as mammalian, bacterial, insect, yeast, and/or plant host cells. In addition, cell-free expression systems may be used. Such expression systems and host cells are standard in the art.

The 2019-nCOV spike protein or immunogenic fragment thereof encoded or expressed (the two terms are used interchangeably herein) by a polynucleotide of the invention typically retain the same binding affinity for its receptor as the native 2019-nCOV spike protein. In the context of the present invention, this may mean having a binding affinity for the 2019-nCOV spike protein receptor of at least 80%, at least 85%, at least 90%, at least 95%, at least 99% or more of that of the native 2019-nCOV spike protein. Preferably the 2019-nCOV spike protein or immunogenic fragment thereof expressed by a polynucleotide of the invention have a binding affinity for the 2019-nCOV spike protein of at least 90%, at least 95%, at least 99% or more of that of the native 2019-nCOV spike protein.

In some embodiments, the 2019-nCOV spike protein or immunogenic fragment thereof expressed by a polynucleotide of the invention have a binding affinity for the 2019-nCoV spike protein receptor greater than that of the full-length protein. For example, the 2019-nCOV spike protein or immunogenic fragment thereof expressed by a polynucleotide of the invention of the invention may have a binding affinity of at least 100%, at least 110%, at least 120%, or at least 150% or more of that of the native 2019-nCOV spike protein.

In other embodiments, the 2019-nCOV spike protein or immunogenic fragment thereof expressed by a polynucleotide of the invention may have a binding affinity for the 2019-nCOV spike protein receptor less than that of the native 2019-nCOV spike protein. For example, the 2019-nCOV spike protein or immunogenic fragment thereof expressed by a polynucleotide of the invention may have a binding affinity of less than 80%, less than 70%, less than 60%, less than 50% or less of that of the native 2019-nCOV spike protein.

The binding affinity of a 2019-nCOV spike protein or immunogenic fragment thereof expressed by a polynucleotide of the invention for its receptor may be quantified in terms of dissociation constant (K_{d}). K_{d} may be determined using any appropriate technique, but SPR is generally preferred in the context of the present invention.

An immunogenic fragment of the 2019-nCOV spike protein expressed by a polynucleotide of the invention are typically greater than 200 amino acids in length. 2019-nCOV spike protein fragments of the present invention may comprise or consist of at least 200, at least 300, at least 400, at least 500, at least 600, at least 700, at least 800, at least 900, at least 1000, at least 1100, or more amino acid residues in length. The fragments of the invention have a common antigenic cross-reactivity with the 2019-nCOV spike protein. In some preferred embodiments, the immunogenic fragment of the 2019-nCOV spike protein expressed by a polynucleotide of the invention is an RBD of the 2019-nCOV spike protein as defined herein, preferably wherein said RBD has at least 90% identity with SEQ ID NO: 15.

The 2019-nCOV spike protein or immunogenic fragment thereof expressed by a polynucleotide of the invention may additionally comprise a leader sequence, for example to assist in the recombinant production and/or secretion of the 2019-nCOV spike protein or immunogenic fragment thereof. Any suitable leader sequence may be used, including conventional leader sequences known in the art. Suitable leader sequences include Bip leader sequences, which are commonly used in the art to aid secretion from insect cells and human tissue plasminogen activator leader sequence (tPA), which is routinely used in viral and DNA based vaccines and for protein vaccines to aid secretion from mammalian cell expression platforms.

The 2019-nCOV spike protein or immunogenic fragment thereof expressed by a polynucleotide of the invention may additionally comprise an N- or C-terminal tag, for example to assist in the recombinant production and/or purification of the 2019-nCOV spike protein or immunogenic fragment thereof. Any N- or C-terminal tag may be used, including conventional tags known in the art. Suitable tags sequences include C-terminal hexa-histidine tags and the “C-tag” (the four amino acids EPEA at the C-terminus), which are commonly used in the art to aid purification from heterologous expression systems, e.g. insect cells, mammalian cells, bacteria, or yeast. In other embodiments, the 2019-nCOV spike protein or immunogenic fragment thereof expressed by a polynucleotide of the invention are purified from heterologous expression systems without the need to use a purification tag.

The 2019-nCOV spike protein or immunogenic fragment thereof expressed by a polynucleotide of the invention may comprise a leader sequence and/or a tag as defined herein.

### Viral Vectors, DNA Plasmids and RNA Vaccines

The present invention also provides a vector: (a) comprising a polynucleotide of the invention; and/or (b) encoding a 2019-nCOV spike protein or immunogenic fragment thereof of the invention. The vector(s) may be present in the form of a vaccine composition or formulation.

The vector of the invention typically expresses a spike protein from 2019-nCOV having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably a vector of the invention expresses a spike protein from 2019-nCOV having at least 90%, at least 95%, at least 96%, at least 97%, at least 98%, at least 99% or more identity with SEQ ID NO: 1, or a fragment thereof, that has a common antigenic cross-reactivity with said spike protein. More preferably, a vector of the invention expresses a spike protein from 2019-nCOV having least 98%, at least 99% or more with SEQ ID NO: 1, or a fragment thereof, that has a common antigenic cross-reactivity with said spike protein. A vector of the invention may express a spike protein from 2019-nCOV comprising or consisting of SEQ ID NO: 1, or a fragment thereof, that has a common antigenic cross-reactivity with said spike protein. In some preferred embodiments, the immunogenic fragment of the 2019-nCOV spike protein expressed by a vector of the invention is an RBD of the 2019-nCOV spike protein as defined herein, preferably wherein said RBD has at least 90% identity with SEQ ID NO: 15.

The vector of the invention may express a spike protein or immunogenic fragment thereof as defined herein which further comprises a signal peptide. Typically said signal peptide directs secretion of the 2019-nCOV spike protein or fragment thereof from a host cell of interest, such as a human cell, an E. coli cell or a yeast cell.

The vector of the invention may further express one or more additional antigen or a fragment thereof. The spike protein or fragment thereof and the one or more additional antigen or fragment thereof may be expressed as a fusion protein. Alternatively, separate vectors expressing the 2019-nCOV spike protein or fragment thereof and the one or more additional antigen or fragment thereof may be used. In such instances, said separate vectors may be used in combination, either sequentially or simultaneously. The one or more additional antigen may be the same antigen or a different antigen from 2019-nCOV, or a fragment thereof. More preferably, said one or more additional antigen is a different antigen from 2019-nCOV, such as an antigen from the 2019-CoV membrane protein or envelope protein.

The vector(s) of the invention may comprise any polynucleotide or expression construct as defined herein, or any combination thereof.

The vector(s) may be a viral vector. Such a viral vector may be an adenovirus (of a human serotype such as AdHu5, a simian serotype such as ChAd63, ChAdOX1 or ChAdOX2, or another form), an adeno-associated virus (AAV), or a poxvirus vector (such as a modified vaccinia Ankara (MVA)), or an adeno associated virus (AAV). ChAdOX1 and ChAdOX2 are disclosed in WO2012/172277 (herein incorporated by reference in its entirety). ChAdOX2 is a BAC-derived and E4 modified AdC68-based viral vector. Preferably said viral vector is an AAV vector adenovirus.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, modified vaccinia Ankara (MVA) can be grown in CEF cells. By way of non-limiting example, the vector may be selected from a human or simian adenovirus or a poxvirus vector.

Typically, the viral vector is incapable of causing a significant infection in an animal subject, typically in a mammalian subject such as a human or other primate.

The vector(s) may be a DNA vector, such as a DNA plasmid. The vector(s) may be an RNA vector, such as a mRNA vector or a self-amplifying RNA vector. The DNA and/or RNA vector(s) of the invention may be capable of expression in eukaryotic and/or prokaryotic cells, particularly any host cell type described herein, or in a subject to be treated.

Typically the DNA and/or RNA vector(s) are capable of expression in a human, E. coli or yeast cell.

The present invention may be a phage vector, such as an AAV/phage hybrid vector as described in Hajitou et al., Cell 2006; 125(2) pp. 385-398; herein incorporated by reference.

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

Vector(s) of the present invention may be designed in silico, and then synthesised by conventional polynucleotide synthesis techniques.

### Virus-Like Particles

Virus-like particles (VLPs) are particles which resemble viruses but do not contain viral nucleic acid and are therefore non-infectious. They commonly contain one or more virus capsid or envelope proteins which are capable of self-assembly to form the VLP. VLPs have been produced from components of a wide variety of virus families (Noad and Roy (2003), Trends in Microbiology, 11:438-444; Grgacic et al., (2006), Methods, 40:60-65). Some VLPs have been approved as therapeutic vaccines, for example Engerix-B (for hepatitis B), Cervarix and Gardasil (for human papilloma viruses).

Accordingly, the invention provides a VLP comprising a 2019-nCOV spike protein or immunogenic fragment thereof of the invention. A VLP of the invention typically comprises a spike protein from 2019-nCOV having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably a VLP of the invention comprises a spike protein from 2019-nCOV having at least 90%, at least 95%, at least 96%, at least 97%, at least 98%, at least 99% or more identity with SEQ ID NO: 1, or a fragment thereof, that has a common antigenic cross-reactivity with said spike protein. More preferably, a VLP of the invention comprises a spike protein from 2019-nCOV having least 98%, at least 99% or more with SEQ ID NO: 1, or a fragment thereof, that has a common antigenic cross-reactivity with said spike protein. A VLP of the invention may comprise a spike protein from 2019-nCOV comprising or consisting of SEQ ID NO: 1, or a fragment thereof, that has a common antigenic cross-reactivity with said spike protein. In some preferred embodiments, the immunogenic fragment of the 2019-nCOV spike protein comprised in a VLP of the invention is an RBD of the 2019-nCOV spike protein as defined herein, preferably wherein said RBD has at least 90% identity with SEQ ID NO: 15.

The skilled person will understand that VLPs can be synthesized through the individual expression of viral structural proteins, which can then self-assemble into the virus-like structure. Combinations of structural capsid proteins from different viruses can be used to create recombinant VLPs. In additions, antigens or immunogenic fragments thereof can be fused to the surface of VLPs. By way of non-limiting example, antigens or immunogenic fragments thereof of the invention may be coupled to a VLP using the SpyCatcher-SpyTag system (as described by Brune, Biswas, Howarth).

A VLP of the invention may comprise one or more additional protein antigen. The one or more additional antigen may be the same antigen or a different antigen from 2019-nCOV, or a fragment thereof. More preferably, said one or more additional antigen is a different antigen from 2019-nCOV, such as an antigen from the 2019-COV membrane protein or envelope protein.

A VLP of the invention may comprise a fusion protein as described herein. A VLP of the invention may comprise a fusion protein of the 2019-nCOV spike protein or immunogenic fragment thereof with Hepatitis B surface antigen (HBSAg), human papillomavirus (HPV) 18 L1 protein, HPV 16 L1 protein and/or Hepatitis E P239, preferably Hepatitis B surface antigen. Although these other viral proteins have been described in fusion proteins previous, to-date, there are no reports of fusion proteins being successfully generated comprising proteins of the size of 2019-nCOV spike protein. Furthermore, there are known limitations regarding the choice of expression systems for such fusion proteins. The present inventors have surprisingly demonstrated that VLPs/fusion proteins comprising 2019-nCOV spike protein can be produced recombinantly in E. coli, yeast and human cells, and that these VLPs/fusion proteins can elicit an (immunoprotective) antibody response in animal models.

Thus, a VLP of the invention may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NOs: 3, 5, 6 or 8. Preferably a VLP of the invention may be encoded by a polynucleotide which comprises or consists of a nucleic acid sequence having at least 90%, at least 95%, at least 96%, at least 97%, at least 98%, at least 99% or more identity to any one of SEQ ID NOs: 3, 5, 6 or 8. More preferably, a VLP of the invention may be encoded by a polynucleotide which comprises or consists of a nucleic acid sequence having at least 98%, at least 99% or more identity to any one of SEQ ID NOs: 3, 5, 6 or 8. A VLP of the invention may be encoded by a polynucleotide which comprises or consists of a nucleic acid sequence of any one of SEQ ID NOS: 3, 5, 6 or 8.

A VLP of the invention may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NOs: 26, 27, 29,30, or 32. Preferably a VLP of the invention may be encoded by a polynucleotide which comprises or consists of a nucleic acid sequence having at least 90%, at least 95%, at least 96%, at least 97%, at least 98%, at least 99% or more identity to any one of SEQ ID NOs: 26, 27, 29, 30, or 32. More preferably, a VLP of the invention may be encoded by a polynucleotide which comprises or consists of a nucleic acid sequence having at least 98%, at least 99% or more identity to any one of SEQ ID NOs: 26, 27, 29, 30, or 32. A VLP of the invention may be encoded by a polynucleotide which comprises or consists of the nucleic acid sequence of any one of SEQ ID NOs: 26, 27, 29, 30, or 32.

A VLP of the invention may comprise or consist of an amino acid sequence having at least 70%, at least 75%, at least 80%, at least 90%, at least 95%, at least 96%, at least 97%, at least 98%, at least 99% or more identity to any one of SEQ ID NO: 9, 10, 11 or 12. Preferably a VLP of the invention may comprise or consist of an amino acid sequence having at least 90%, at least 95%, at least 96%, at least 97%, at least 98%, at least 99% or more identity to any one of SEQ ID NOs: 9, 10, 11 or 12. More preferably, a VLP of the invention may comprises or consists of an amino acid sequence having at least 98%, at least 99% or more identity to any one of SEQ ID NOs: 9, 10, 11 or 12. A VLP of the invention may comprise or consist of an amino acid sequence of any one of SEQ ID NOs: 9, 10, 11 or 12.

A VLP of the invention may comprise or consist of an amino acid sequence having at least 70%, at least 75%, at least 80%, at least 90%, at least 95%, at least 96%, at least 97%, at least 98%, at least 99% or more identity to any one of SEQ ID NOs: 28,31, or 33. Preferably a VLP of the invention may comprise or consist of an amino acid sequence having at least 90%, at least 95%, at least 96%, at least 97%, at least 98%, at least 99% or more identity to any one of SEQ ID NOs: 28, 31, or 33. More preferably, a VLP of the invention may comprises or consists of an amino acid sequence having at least 98%, at least 99% or more identity to any one of SEQ ID NOs: 28, 31, or 33. A VLP of the invention may comprise or consist of an amino acid sequence of any one of SEQ ID NOs: 28, 31, or 33.

The use of VLP may increase the efficacy of the immunoprotective response induced by the 2019-nCOV spike protein or immunogenic fragment and/or may increase the duration of the immunoprotective response as defined herein.

### Fusion Proteins

The invention further provides a fusion protein comprising a 2019-nCOV spike protein or immunogenic fragment thereof of the invention. A fusion protein of the invention typically comprises a spike protein from 2019-nCOV having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably a fusion protein of the invention comprises a spike protein from 2019-nCOV having at least 90%, at least 95%, at least 96%, at least 97%, at least 98%, at least 99% or more identity with SEQ ID NO: 1, or a fragment thereof, that has a common antigenic cross-reactivity with said spike protein. More preferably, a fusion protein of the invention comprises a spike protein from 2019-nCOV having least 98%, at least 99% or more with SEQ ID NO: 1, or a fragment thereof, that has a common antigenic cross-reactivity with said spike protein. A fusion protein of the invention may comprise a spike protein from 2019-nCOV comprising or consisting of SEQ ID NO: 1, or a fragment thereof, that has a common antigenic cross-reactivity with said spike protein. In some preferred embodiments, the immunogenic fragment of the 2019-nCOV spike protein comprised in a fusion protein of the invention is an RBD of the 2019-nCOV spike protein as defined herein, preferably wherein said RBD has at least 90% identity with SEQ ID NO: 15.

A fusion protein of the invention typically also comprises a non-2019-nCOV domain or element, typically a non-2019-nCOV protein, polypeptide or peptide domain or element. A fusion protein of the invention may comprise the 2019-nCOV spike protein or immunogenic fragment thereof and one or more of: Hepatitis B surface antigen (HBSAg); human papillomavirus (HPV) 18 L1 protein; HPV 16 L1 protein; and/or Hepatitis E P239 (HEV), preferably Hepatitis B surface antigen. As described above in the context of VLPs, the present inventors have surprisingly demonstrated that fusion proteins comprising 2019-nCOV spike protein can be produced recombinantly in E. coli, yeast and human cells, and that these fusion proteins can elicit an (immunoprotective) antibody response in animal models.

A fusion protein of the invention may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NO: 3, 5, 6 or 8. Preferably a fusion protein of the invention may be encoded by a polynucleotide which comprises or consists of a nucleic acid sequence having at least 90%, at least 95%, at least 96%, at least 97%, at least 98%, at least 99% or more identity to any one of SEQ ID NOs: 3, 5, 6 or 8. More preferably, a fusion protein of the invention may be encoded by a polynucleotide which comprises or consists of a nucleic acid sequence having at least 98%, at least 99% or more identity to any one of SEQ ID NOs: 3, 5, 6 or 8. A VLP of the invention may be encoded by a polynucleotide which comprises or consists of a nucleic acid sequence of any one of SEQ ID NOs: 3, 5, 6 or 8.

A fusion protein of the invention may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NOs: 26, 27, 29,30, or 32. Preferably a fusion protein of the invention may be encoded by a polynucleotide which comprises or consists of a nucleic acid sequence having at least 90%, at least 95%, at least 96%, at least 97%, at least 98%, at least 99% or more identity to any one of SEQ ID NOs: 26, 27, 29, 30, or 32. More preferably, a fusion protein of the invention may be encoded by a polynucleotide which comprises or consists of a nucleic acid sequence having at least 98%, at least 99% or more identity to any one of SEQ ID NOs: 26, 27, 29, 30, or 32. A fusion protein of the invention may be encoded by a polynucleotide which comprises or consists of the nucleic acid sequence of any one of SEQ ID NOs: 26, 27, 29, 30, or 32.

A fusion protein of the invention may comprise or consist of an amino acid sequence having at least 70%, at least 75%, at least 80%, at least 90%, at least 95%, at least 96%, at least 97%, at least 98%, at least 99% or more identity to any one of SEQ ID NO: 9, 10, 11 or 12. Preferably a fusion protein of the invention may comprise or consist of an amino acid sequence having at least 90%, at least 95%, at least 96%, at least 97%, at least 98%, at least 99% or more identity to any one of SEQ ID NOs: 9, 10, 11 or 12. More preferably, a fusion protein of the invention may comprises or consists of an amino acid sequence having at least 98%, at least 99% or more identity to any one of SEQ ID NOs: 9, 10, 11 or 12. A fusion protein of the invention may comprise or consist of an amino acid sequence of any one of SEQ ID NOs: 9, 10, 11 or 12.

A fusion protein of the invention may comprise or consist of an amino acid sequence having at least 70%, at least 75%, at least 80%, at least 90%, at least 95%, at least 96%, at least 97%, at least 98%, at least 99% or more identity to any one of SEQ ID NOs: 28, 31, or 33. Preferably a fusion protein of the invention may comprise or consist of an amino acid sequence having at least 90%, at least 95%, at least 96%, at least 97%, at least 98%, at least 99% or more identity to any one of SEQ ID NOs: 28, 31, or 33. More preferably, a fusion protein of the invention may comprise or consist of an amino acid sequence having at least 98%, at least 99% or more identity to any one of SEQ ID NOs: 28, 31, or 33. A fusion protein of the invention may comprise or consist of an amino acid sequence of any one of SEQ ID NOs: 28, 31, or 33.

A fusion protein of the invention may comprise a linker (also referred to interchangeably herein as a linker peptide, a spacer or a spacer peptide). A linker may be used to join two or more functional domains of a fusion protein of the invention. Typically, where a linker is present, it is used to join the 2019-nCOV spike protein or immunogenic fragment thereof domain of the fusion protein to the non-2019-nCOV spike protein domain of the fusion protein. Use of linkers in fusion proteins is routine in the art, and any conventional linker protein may be used in fusion proteins of the invention, provided that the resulting fusion protein retains the desired functional properties of the 2019-nCOV spike protein or immunogenic fragment thereof and the desired function properties of the non-2019-nCOV spike protein domain.

A linker may be a short peptide of up to about 30 amino acids, such as about 5-30 amino acids, about 5-25 amino acids, about 5-20 amino acids, about 10-20 amino acids, about 5-15 amino acids or about 10-15 amino acids in length. In some embodiments, the linker is about 10, about 11, about 12, about 13, about 14, about 15, about 16, about 17, about 18, about 19 or about 20 amino acids in length.

In some embodiments a rigid linker may be used in fusion proteins of the invention. Rigid linkers are conventionally used when it is necessary to keep a fixed distance between the different domains/portions of a fusion protein and to maintain their independent functions. Rigid linkers may also be used when the spatial separation of the fusion protein domains is critical to preserve the stability or bioactivity of the fusion proteins. An empirical rigid linker with the sequence of A(EAAAK)ₙA (n=2-5) (SEQ ID NO: 16) displayed α-helical conformation, which is stabilized by Glu⁻Lys⁺ salt bridges. The length of linker may be adjusted by changing the copy number to achieve an optimal distance between domains. Helical linkers can also improve fusion protein folding and stability.

A non-limiting example of a rigid linker is EAAAKEAAAKEAAAK (also referred to as (EAAAK)₃, SEQ ID NO: 18), which may be encoded by the nucleic acid sequence (SEQ ID NO: 17). The length and structure of the linkers, controlling the distance between functional domains, affect the stability of the fusion protein. The stability and activity of the RBD fusion proteins of the invention are typically significantly improved after (EAAAK)₃ linker insertion, as shown in the Examples herein. Use of linkers, particularly (EAAAK)₃, may also enhance yield of the recombinant fusion proteins of the invention.

Accordingly, rigid linkers, particularly (EAAAK)₃ (SEQ ID NO: 18), may be preferably used for expression of fusion proteins of the invention in mammalian cells, such as HEK 293 cells.

In some embodiments, flexible linkers may be used in fusion proteins of the invention. Flexible linkers are conventionally used when the joined domains require a certain degree of movement or interaction. Flexible linkers usually comprise or consist of small amino acid residues, such as glycine, threonine, arginine, serine, asparagine, glutamine, alanine, aspartic acid, proline, glutamic acid, lysine, leucine and/or valine, particularly glycine, serine, alanine, leucine and/or valine. Flexible linkers comprising or consisting of glycine, serine and/or alanine are preferred, with glycine and serine being particularly preferred. Accordingly, the most commonly used flexible linkers have sequences consisting primarily of stretches of Gly and Ser residues (“GS” linker), which comprise a sequence of (Gly-Gly-Gly-Gly-Ser)ₙ (SEQ ID NO: 19). Non-limiting examples of GS linkers include GS5 or (GGGGS): (SEQ ID NO: 20); GS10 or (GGGGS)₂ (SEQ ID NO: 21); GS15 or (GGGGS)₃ (SEQ ID NO: 23); GS20 or (GGGGS)₄ (SEQ ID NO: 24); and GS25 or(GGGGS)₅ (SEQ ID NO: 25). Preferably, GS15 may be used, which may be encoded by (SEQ ID NO: 22). Flexible linkers may be preferably used for expression of fusion proteins of the invention in bacterial cells, such as E. coli cells.

Any appropriate linker, such as the exemplary linkers described herein may be used with any fusion protein of the invention (comprising any 2019-nCOV spike protein or immunogenic fragment domain and any non-219-nCOV spike protein domain). By way of non-limiting example, a fusion protein of the invention may comprise or consist of HBSAg-(EAAAK)₃-RBD (SEQ ID NO: 28), or a variant with at least 90% sequence identity thereto, which may be encoded by SEQ ID NO: 26 or 27, or a variant with at least 90% sequence identity thereto. By way of a further non-limiting example, a fusion protein of the invention may comprise or consist of HBSAg-(EAAAK)₃-full-length 2019-nCOV spike protein (SEQ ID NO: 33), or a variant with at least 90% sequence identity thereto, which may be encoded by SEQ ID NO: 32, or a variant with at least 90% sequence identity thereto. By way of further non-limiting example, a fusion protein of the invention may comprise or consist of HEV-GS15-RBD (SEQ ID NO: 31), or a variant with at least 90% sequence identity thereto, which may be encoded by (SEQ ID NO: 29 or 30), or a variant with at least 90% sequence identity thereto.

A fusion protein of the invention may preferably take the form of a VLP. Without being bound by theory, this is because HBSAg, HPV 18 L1 protein, HPB 16 L1 protein and Hepatitis E P239 protein are known to spontaneously form VLPs when expressed recombinantly, and this structure is retained when HBSAg, HPV 18 L1 protein, HPB 16 L1 protein and/or Hepatitis E P239 protein are present in fusion protein form combined with a 2019-nCOV spike protein of the invention (or immunogenic fragment thereof).

### Antibodies

As described herein, the 2019-nCOV spike protein or fragment thereof encoded by a polynucleotide of the invention elicit the production of antibodies which specifically bind to the one or more conformational epitope found in native 2019-nCOV. Said antibodies are typically neutralising antibodies (nAb) as discussed below. These nAb are capable of mediating an immunoprotective effect against 2019-nCoV.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody entities are known in the art, non-limiting embodiments of which are discussed below.

In a full-length antibody, each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Antibodies may be polyclonal (pAb) or monoclonal (mAb). When used therapeutically (i.e. to provide passive immunity), the administration of mAbs is preferred.

According to the invention, antibodies can be of any type (e.g., IgG, IgE, IgM, IgD, IgA and IgY), class (e.g., IgG1, IgG2, IgG3, IgG4, IgA1 and IgA2) or subclass and may be from any species (e.g., mouse, human, chicken, rat, rabbit, sheep, shark and camelid).

The term “antigen-binding fragment” of an antibody (or simply “binding fragment”), as used herein, refers to one or more fragments of an antibody that retain the ability to specifically bind to an antigen. It has been shown that the antigen-binding function of an antibody can be performed by one or more fragments of a full-length antibody. Single chain antibodies are also encompassed. Such antigen-binding fragments may also be bispecific, dual specific, or multi-specific, specifically binding to two or more different antigens. Thus, examples of binding fragments encompassed within the term “antigen-binding fragment” of an antibody include Fab, Fv, scFv, dAb, Fd, Fab′ or F(ab′)₂, tandem scFv and diabodies.

Also encompassed are antibody constructs, defined as a polypeptide comprising one or more the antigen binding fragment of the invention linked to a linker polypeptide or an immunoglobulin constant domain. Linker polypeptides comprise two or more amino acid residues joined by peptide bonds and are used to link one or more antigen binding portions.

The term “antibody” as used herein may be a human antibody; defined as an antibody having variable and constant regions derived from human germline immunoglobulin sequences, but which may include amino acid residues not encoded by human germline immunoglobulin sequences (e.g., mutations introduced by random or site-specific mutagenesis in vitro or by somatic mutation in vivo), for example in the CDRs and in particular CDR3. Recombinant human antibodies are also encompassed.

An antibody of the invention may be a “chimeric antibody”; defined as an antibody which comprises heavy and light chain variable region sequences from one species and constant region sequences from another species. The present invention encompasses chimeric antibodies having, for example, murine heavy and light chain variable regions linked to human constant regions.

An antibody of the invention may be a “CDR-grafted antibody”; defined as an antibody which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3 or all three CDRs) has been replaced with human CDR sequences.

An antibody of the invention may be a “humanized antibody”; defined as an antibody which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

The terms “Kabat numbering”, “Kabat definitions and “Kabat labelling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and Kabat, E. A., et al. (1991) Sequences of Proteins of Immunological Interest, Fifth Edition, U.S. Department of Health and Human Services, NIH Publication No. 91-3242).

Antibodies of the invention are not limited to a particular method of generation or production. Thus, the invention provides antibodies which have been manufactured from a hybridoma that secretes the antibody, as well as antibodies produced from a recombinantly produced cell that has been transformed or transfected with a polynucleotide or polynucleotides encoding the antibody. Such hybridomas, recombinantly produced cells, and polynucleotides form part of the invention.

An antibody, or antigen-binding fragment thereof, of the invention is selective or specific for the 2019-nCOV spike protein, or a particular epitope (preferably a conformational epitope) of the 2019-nCOV spike protein as described herein. By specific, it will be understood that the antibody binds to the molecule of interest, in this case the 2019-nCOV spike protein or fragment thereof, with no significant cross-reactivity to any other molecule, particularly any other protein. For example, a binding compound or antibody of the invention that is specific for a particular 2019-nCOV spike protein epitope of the invention will show no significant cross-reactivity with other 2019-nCOV spike protein epitopes. As another example, a binding compound or antibody of the invention that is specific for the 2019-nCOV spike protein will show no significant cross-reactivity with the 2019-nCOV membrane protein. Cross-reactivity may be assessed by any suitable method. Cross-reactivity of a binding compound (e.g. antibody) for a 2019-nCOV spike protein epitope with another 2019-nCOV spike protein epitope or a protein other than 2019-nCOV spike protein may be considered significant if the binding compound (e.g. antibody) binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the 2019-nCOV spike protein epitope. A binding compound (e.g. antibody) that is specific for the 2019-nCOV spike protein or fragment thereof may bind to another molecule such as 2019-nCOV membrane protein at less than 90%, 85%, 80%, 75%, 70%, 65%, 60%, 55%, 50%, 45%, 40%, 35%, 30%, 25% or 20% the strength that it binds to the 2019-nCOV spike protein epitope. Preferably, the binding compound (e.g. antibody) binds to the other molecule at less than 20%, less than 15%, less than 10% or less than 5%, less than 2% or less than 1% the strength that it binds to the 2019-nCOV spike protein epitope. Binding affinity may be quantified in any suitable way, e.g. by K_{D}.

The binding affinity of a 2019-nCOV spike protein antibody (preferably neutralising) of the invention for 2019-nCOV spike protein may be quantified in terms of dissociation constant (K_{D}). K_{D} may be determined using any appropriate technique, but SPR is generally preferred in the context of the present invention. A 2019-nCOV spike protein antibody of the invention may bind to 2019-nCOV spike protein with a K_{D} of less than 1 μM, less than 100 nM, less than 50 nM, less than 25 nM, less than 10 nM, less than 1 nM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 10 pM, less than 5 pM, or less. Typically a 2019-nCOV spike protein antibody of the invention binds to 2019-nCOV spike protein with a K_{D} of less 50 nM, less than 10 nM or less than 1 nM.

Further antibodies which bind to the epitopes/antigens of the invention may be generated by producing variants of the antibodies of the invention. Such variants may have CDRs sharing a high level of identity with the CDRs of an antibody of the invention, for example may have CDRs each of which independently may differ by one or two amino acids from the antibody of the invention from which the variant antibody is derived, and wherein the variant retains the binding and functional properties of the antibody of the invention. Additionally, such antibodies may have one or more variations (e.g. a conservative amino acid substitution) in the framework regions. The variations in the amino acid sequences of the antibodies of the invention should maintain at least 75%, at least 80%, at least 85%, at least 90%, at least 95% and up to 99% sequence identity. Variants having at least 90% sequence identity with the antibodies of the invention, particularly the specific antibodies exemplified herein are specifically contemplated. Variation may or may not be limited to the framework regions and not present in the CDRs.

The term “neutralising antibody” is defined herein to mean an antibody which by itself (in the absence of any other 2019-nCOV spike protein antibody or other antibody against another 2019-nCOV protein) has the ability to affect the function of the spike protein to which it binds. In particular, neutralising antibodies reduce the ability of 2019-nCOV viral particles expressing the spike protein from infecting a cell by neutralising or inhibiting the biological activity of the spike protein.

This neutralising activity may be quantified using any appropriate technique and measured in any appropriate units. This disclosure applies equally to neutralising antibodies of the invention (and to other binding compounds as described herein). For example, the effectiveness of a 2019-nCOV spike protein or immunogenic fragment thereof may be given in terms of their half maximal effective concentration (EC50), antibody titre stimulated (in terms of antibody units, AU) and/or EC50 in terms of AU. The latter of these gives an indication of the quality of the antibody response stimulated by the 2019-nCOV spike protein or immunogenic fragment thereof of the invention. Any appropriate technique may be used to determine the EC50, AU or EC50/AU. Conventional techniques are known in the art.

The amount of antibody produced may be quantified using any appropriate method, with standard techniques being known in the art. For example, the amount of antibody produced may be measured by ELISA in terms of the serum IgG response induced by the 2019-nCOV spike protein or immunogenic fragment thereof of the invention. The amount of antibody produced may be given in terms of arbitrary antibody units (AU).

The immune response (or immunogenicity) to a 2019-nCOV spike protein or immunogenic fragment thereof of the invention, particularly the antibody response, may be given as the half-maximal effective concentration in terms of the amount of antibody produced, i.e. EC50/AU. This gives an indication of the quality of the immune response generated to the 2019-nCOV spike protein or immunogenic fragment thereof. For example, a low EC50 (i.e. effective response) but a high number of antibody units generated is less effective (and gives a higher EC50/AU) than a low EC50 with a low number of antibody units. This value thus indicates the quality of the antibody response by representing the neutralising antibody activity (measured as the EC50) as a proportion of the total amount of anti-2019-nCOV spike protein or immunogenic fragment thereof IgG antibody produced (measured by ELISA in AU). A more effective vaccine thus induces the EC50 with less antibody (lower AU).

Typically, the neutralising 2019-nCOV spike protein antibodies of the invention reduce the infectivity of 2019-nCOV particles by at least at least 40%, at least 50%, at least 60%, at least 70%, at least 80%, at least 90% or more.

A 2019-nCOV spike protein or immunogenic fragment thereof of the invention may elicit an improved immune response, particularly an improved antibody response, compared with the native 2019-nCOV spike protein or immunogenic fragment thereof or a 2019-nCOV spike protein or immunogenic fragment thereof produced by a non-optimised polynucleotide.

Any and all disclosure herein in relation to binding compounds preferably relates to antibodies as described herein.

Alternatively, other binding compounds, such as DNA oligonucleotide aptamers, RNA oligonucleotide aptamers, and other engineered biopolymers against 2019-nCOV spike protein or a fragment thereof, particularly an epitope within said 2019-nCOV spike protein or fragment, may also be able to replicate the activity of the antibodies and combinations thereof described here. Said alternative binding compounds may specifically bind to the protein or immunogenic fragment thereof of the invention.

Oligonucleotide aptamers may be identified or synthesised using well-established methods. The aptamer may further me optimised to render is suitable for therapeutic use, e.g. it may be conjugated to a monoclonal antibody to modify its pharmacokinetics and/or recruit Fc-dependent immune functions.

### Compositions and Therapeutic Indications

As described herein, the present inventors have demonstrated that immunisation with 2019-nCOV spike protein expressed by polynucleotides of the invention are able to generate a robust antibody response.

Accordingly, the present invention provides a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, or a binding compound of the invention for use as a vaccine.

The invention also provides a vaccine composition comprising said polynucleotide, expression construct, viral vector, DNA plasmid or RNA vaccine which expresses a 2019-nCOV spike protein or immunogenic fragment thereof, or a 2019-nCOV spike protein or immunogenic fragment thereof, or a binding compound. The vaccine composition may optionally comprise a pharmaceutically acceptable excipient, diluent, carrier, propellant, salt and/or additive.

In some embodiments the vaccine composition comprises at least two different proteins or immunogenic fragments according to the invention, and/or at least two different polynucleotide molecules according to the invention. By way of non-limiting example, the vaccine composition may comprise a polynucleotide encoding a 2019-nCOV spike protein and a polynucleotide encoding a 2019-nCOV membrane protein.

The present invention also provides a method of stimulating or inducing an immune response in a subject using a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, or a binding compound of the invention (as described above).

Said method of stimulating or inducing an immune response in a subject may comprise administering a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, or a binding compound of the invention (as described above) to a subject.

In the context of the therapeutic uses and methods, a “subject” is any animal subject that would benefit from stimulation or induction of an immunoprotective response against 2019-nCoV. Typical animal subjects are mammals, such as primates, for example, humans.

Thus, the present invention provides a method for treating or preventing 2019-nCOV infection. Said method typically comprises the administration of a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, a vaccine composition or a binding compound of the invention to a subject in need thereof.

The present invention also provides a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, a vaccine composition or a binding compound of the invention for use in prevention or treatment of 2019-nCoV infection.

The present invention also provides the use of a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, a vaccine composition or a binding compound of the invention for the manufacture of a medicament for the prevention or treatment of 2019-nCoV infection.

As used herein, the term “treatment” or “treating” embraces therapeutic or preventative/prophylactic measures, and includes post-infection therapy and amelioration of a 2019-nCoV infection.

As used herein, the term “preventing” includes preventing the initiation of infection by 2019-nCOV and/or reducing the severity or intensity of an infection by 2019-nCOV. The term “preventing” includes inducing or providing protective immunity against infection by 2019-nCoV. Immunity to infection by a 2019-nCOV may be quantified using any appropriate technique, examples of which are known in the art.

A polynucleotide, expression construct, viral vector, DNA plasmid or RNA vaccine which expresses a 2019-nCOV spike protein or immunogenic fragment thereof, or a 2019-nCOV spike protein or immunogenic fragment thereof, a vaccine composition or a binding compound defined herein may be administered to a subject (typically a mammalian subject such as a human or other primate) already having a 2019-nCoV infection, a condition or symptoms associated with infection by 2019-nCOV, to treat or prevent infection by 2019-nCOV. For example, the subject may be suspected of having come in contact with 2019-nCOV, or has had known contact with 2019-nCOV, but is not yet showing symptoms of exposure.

When administered to a subject (e.g. a mammal such as a human or other primate) that already has a 2019-nCoV infection, or is showing symptoms associated with a 2019-nCOV infection, the polynucleotide, expression construct, viral vector, DNA plasmid or RNA vaccine which expresses a 2019-nCOV spike protein or immunogenic fragment thereof, or a 2019-nCOV spike protein or immunogenic fragment thereof, a vaccine composition or a binding compound as defined can cure, delay, reduce the severity of, or ameliorate one or more symptoms, and/or prolong the survival of a subject beyond that expected in the absence of such treatment.

Alternatively, a polynucleotide, expression construct, viral vector, DNA plasmid or RNA vaccine which expresses a 2019-nCOV spike protein or immunogenic fragment thereof, or a 2019-nCOV spike protein or immunogenic fragment thereof, a vaccine composition or a binding compound as defined herein may be administered to a subject (e.g. a mammal such as a human or other primate) who ultimately may be infected with 2019-nCOV, in order to prevent, cure, delay, reduce the severity of, or ameliorate one or more symptoms of said 2019-nCoV infection, or in order to prolong the survival of a subject beyond that expected in the absence of such treatment, or to help prevent that subject from transmitting a 2019-nCoV infection.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects. As used herein, the term “preventing” includes preventing the initiation of 2019-nCOV infection and/or reducing the severity or intensity of a 2019-nCoV infection. The term “preventing” includes inducing or providing protective immunity against 2019-nCoV infection. Immunity to 2019-nCOV infection may be quantified using any appropriate technique, examples of which are known in the art.

As used, herein, a “vaccine” is a formulation that, when administered to an animal subject such as a mammal (e.g. a human or other primate) stimulates a protective immune response against 2019-nCoV infection. The immune response may be a humoral and/or cell-mediated immune response. A vaccine of the invention can be used, for example, to protect a subject from the effects of 2019-nCoV infection.

### Pharmaceutical Compositions and Formulations

The term “vaccine” is herein used interchangeably with the terms “therapeutic/prophylactic composition”, “formulation” or “medicament”. The vaccine of the invention (as defined above) can be combined or administered in addition to a pharmaceutically acceptable carrier. Alternatively or in addition the vaccine of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations (e.g. vaccines) is generally by conventional routes e.g. intravenous, subcutaneous, intraperitoneal, or mucosal routes. The administration may be by parenteral injection, for example, a subcutaneous, intradermal or intramuscular injection. Formulations comprising neutralizing antibodies may be particularly suited to administration intravenously, intramuscularly, intradermally, or subcutaneously.

Accordingly, immunogenic compositions, therapeutic formulations, medicaments and prophylactic formulations (e.g. vaccines) of the invention are typically prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as the 2019-nCOV spike proteins, fragments thereof, nucleic acids encoding said spike proteins, expression vectors, virial vectors, DNA plasmids, RNA vaccines, fusion proteins and vaccine compositions) are often mixed with carriers, diluents, excipients or similar which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Generally, the carrier, diluent, excipient or similar is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/ or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, GLA-SE (GLA is a synthetic lipid A derivative, in GLA-SE this is formulated as an oil-in-water emulsion using squalene oil), monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium). Preferred adjuvants include: aluminium hydroxide and aluminium phosphate gel; aluminium hydroxide and monophosphoryl lipid A (MPL); and 5% squalene (MF59). Other preferred adjuvants include alum or aluminium hydroxide and/or aluminium phosphate, GLA-SE and/or a squalene-based adjuvant such as MF59 or AddaVax™, or any combination thereof. Preferably a combination of aluminium hydroxide and/or aluminium phosphate, GLA-SE and AddaVax™ is used.

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

### DEFINITIONS

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

The term “variant”, when used in relation to a protein, means a peptide or peptide fragment of the protein that contains one or more analogues of an amino acid (e.g. an unnatural amino acid), or a substituted linkage.

The term “derivative”, when used in relation to a protein, means a protein that comprises the protein in question, and a further peptide sequence. The further peptide sequence should preferably not interfere with the basic folding and thus conformational structure of the original protein. Two or more peptides (or fragments, or variants) may be joined together to form a derivative. Alternatively, a peptide (or fragment, or variant) may be joined to an unrelated molecule (e.g. a second, unrelated peptide). Derivatives may be chemically synthesized, but will be typically prepared by recombinant nucleic acid methods. Additional components such as lipid, and/or polysaccharide, and/or polypeptide components may be included.

Reference to 2019-nCOV polynucleotides and/or proteins in the present specification embraces fragments and variants thereof. Variant 2019-nCOV spike proteins retain one or more conformational epitope of native spike protein and the ability to elicit the production of neutralising antibodies and/or an immunoprotective response. Variant 2019-nCOV spike protein polynucleotides of the invention encode such spike proteins. By way of example, a variant may have at least 80%, preferably at least 90%, more preferably at least 95%, and most preferably at least 97 or at least 99% amino acid sequence homology with the reference sequence (e.g. a 2019-nCOV polynucleotide and/or protein of the invention, particularly any SEQ ID NO presented in the present specification which defines a 2019-nCOV polynucleotide and/or protein). Thus, a variant may include one or more analogues of a polynucleotide (e.g. an unnatural nucleic acid), or a substituted linkage. Also, by way of example, the term fragment, when used in relation to a 2019-nCOV polynucleotide and/or protein, means a polynucleotide having at least ten, preferably at least fifteen, more preferably at least twenty nucleic acid residues of the reference 2019-nCOV polynucleotide and/or protein. The term fragment also relates to the above-mentioned variants. Thus, by way of example, a fragment of a 2019-nCOV polynucleotide and/or protein of the present invention may comprise a nucleic acid sequence having at least 10, 20 or 30 nucleic acids, wherein the polynucleotide sequence has at least 80% sequence homology over a corresponding nucleic acid sequence (of contiguous) nucleic acids of the reference 2019-nCOV polynucleotide and/or protein sequence. These definitions of fragments and variants also apply to other polynucleotides of the invention. In the context of peptide sequences, the term fragment means a peptide having at least ten, preferably at least fifteen, more preferably at least twenty amino acid residues of the reference protein. The term fragment also relates to the above-mentioned variants. Thus, by way of example, a fragment may comprise an amino acid sequence having at least 10, 20 or 30 amino acids, wherein the amino acid sequence has at least 80% sequence homology over a corresponding amino acid sequence (of contiguous) amino acids of the reference sequence.

Some exemplified polynucleotides herein comprise additional motifs, particularly restriction enzyme sites, KOZAC sequences and/or motifs (e.g. tga taa) to drive protein secretion. One of skill in the art will appreciate that these additional sequences may be omitted. As such, the invention encompasses the exemplified nucleic acid sequences comprising the specified motifs, as well as the nucleic acid sequences lacking one or more of these motifs.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Preferably the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of pain. A subject can be male or female, adult or juvenile.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, a subject can be one who exhibits one or more risk factors for a condition or one or more complications related to said condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A polypeptide, e.g., a fusion polypeptide or portion thereof (e.g. a domain), can be a variant of a sequence described herein. Preferably, the variant is a conservative substitution variant. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wild-type, e.g. 110%, 125%, 150%, 175%, 200%, 500%, 1000% or more.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

A polypeptide as described herein may comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

A polypeptide as described herein may comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). A polypeptide as described herein may comprise alternative amino acids. Non-limiting examples of alternative amino acids include D amino acids, beta-amino acids, homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, paraaminophenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alphaamino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

A polypeptide may be modified, e.g. by addition of a moiety to one or more of the amino acids comprising the peptide. A polypeptide as described herein may comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. A polypeptide as described herein may comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization.

Alterations of the original amino acid sequence can be accomplished by any of a number of techniques known to one of skill in the art. Amino acid substitutions can be introduced, for example, at particular locations by synthesizing oligonucleotides containing a codon change in the nucleotide sequence encoding the amino acid to be changed, flanked by restriction sites permitting ligation to fragments of the original sequence. Following ligation, the resulting reconstructed sequence encodes an analogue having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. A polypeptide as described herein may be chemically synthesized and mutations can be incorporated as part of the chemical synthesis process.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA In another aspect, the nucleic acid can be RNA Suitable nucleic acid molecules are DNA, including genomic DNA or cDNA. Other suitable nucleic acid molecules are RNA, including mRNA.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention.

### SEQUENCE HOMOLOGY

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

### Alignment Score for Determining Sequence Identity

BLOSUM62 TABLE

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 20 | −1 | −3 | 1 | 1 | −2 | −1 | −3 | −2 | 5 | | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 1 | 1 | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the number of \\ gaps introduced into the longer sequence \\ in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
lysine
histidine
   Acidic: glutamic acid
aspartic acid
   Polar: glutamine
asparagine
   Hydrophobic: leucine
isoleucine
valine

Aromatic: phenylalanine
tryptophan
tyrosine
   Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and a -methyl serine) may be substituted for amino acid residues of the polypeptides of the present invention. A limited number of non-conservative amino acids, amino acids that are not encoded by the genetic code, and unnatural amino acids may be substituted for clostridial polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allothreonine, methyl-threonine, hydroxy-ethylcysteine, hydroxyethylhomo-cysteine, nitroglutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90: 10145-9, 1993). In a second method, translation is carried out in Xenopus oocytes by microinjection of mutated mRNA and chemically aminoacylated suppressor tRNAs (Turcatti et al., J. Biol. Chem. 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3- azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241 :53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30: 10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

The following Examples illustrate the invention.

### EXAMPLES

The nucleic acid encoding the 2019-nCOV spike protein (S) has been modified for expression in various expression systems, including—E coli, yeast and human cells to induce neutralizing antibodies response to protect against 2019-nCoV infection.

N and C terminal deleted amino acids (5-10) were cloned to express and refold the S protein in the native conformation of coronavirus in an E. coli system while others (Yeast and Human cells) as whole protein. S protein from coronavirus was also combined and express as fusion proteins with virus like particles of Hepatitis E (P239) and Human Papilloma virus like particle (18L1) in order to increase the efficacy and longevity of the immunoprotective response.

### Example 1: E. coli Based 2019-nCOV Spike Protein Expression

The nucleic acid sequence of coronavirus S protein (Wuhan-Hu-1-CoV-S, GenBank: MN908947.3) was optimised for expression in E coli using Geneart for codon optimisation and containing SacI and NotI single cloning sites to design the nucleic acid sequence of SEQ ID NO: 2.

The codon usage was adapted to the codon bias of Escherichia coli genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content was adjusted (average GC content 45%) to prolong mRNA half life. Codon usage was adapted to the bias of E. coli resulting in a CAI (Codon Adaptation Index) value of 0.96. The optimized gene has been designed to allow high and stable expression rates in E. coli.

### Example 2: 2019-nCOV Spike Protein in Hepatitis E Virus-Like Particles

The nucleic acid sequence of coronavirus S protein was optimised for expression in E. coli and used to generate Hepatitis E Virus-Like Particles comprising the S protein (Wuhan-Hu-1-HEV-CoV-S).

The gene synthesis was codon optimized for expression in E. coli by Geneart with Sacl and NotI single cloning sites to design the nucleic acid sequence of SEQ ID NO: 3.

The codon usage was adapted to the codon bias of Escherichia coli genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content (46%) was adjusted to prolong mRNA half life. Codon usage was adapted to the bias of E. coli resulting in a CAI value of 0.96. The optimized gene has been designed to allow high and stable expression rates in E. coli.

### Example 3: Komagataella pastoris Based 2019-nCOV Spike Protein Expression

The nucleic acid sequence of coronavirus S protein (Wuhan-Hu-1 CoV-S) was optimised for expression in K. pastoris using Geneart for codon optimisation and containing BstBI-NotI single cloning sites to design the nucleic acid sequence of SEQ ID NO: 4.

The codon usage was adapted to the codon bias of K. pastoris genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content (48%) was adjusted to prolong mRNA half life. Codon usage was adapted to the bias of K. pastoris resulting in a CAI value of 0.84. The optimized gene has been designed to allow high and stable expression rates in K. pastoris.

### Example 4: Expression of a Fusion Protein Comprising 2019-nCOV Spike Protein in K. pastoris

The nucleic acid sequence of coronavirus S protein (Wuhan-Hu-1-HPV18 L1-CoV-S) was optimised for expression in K. pastoris as a fusion protein with HPV 18 L1. BstBI and NotI are single cloning sites to design the nucleic acid sequence of SEQ ID NO: 5

The codon usage was adapted to the codon bias of K. pastoris genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content (48%) was adjusted to prolong mRNA half life. Codon usage was adapted to the bias of K. pastoris resulting in a CAI value of 0.84. The optimized gene has been designed to allow high and stable expression rates in K. pastoris.

### Example 5: Expression of a Fusion Protein Comprising 2019-nCOV Spike Protein in K. pastoris

The nucleic acid sequence of coronavirus S protein (Wuhan-Hu-1-HPV16 L1-CoV-S) was optimised for expression in K. pastoris as a fusion protein with HPV 16 L1. BstBI and NotI are single cloning sites to design the nucleic acid sequence of SEQ ID NO: 6

The codon usage was adapted to the codon bias of K. pastoris genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content (48%) was adjusted to prolong mRNA half life. Codon usage was adapted to the bias of K. pastoris resulting in a CAI value of 0.84. The optimized gene has been designed to allow high and stable expression rates in Pichia pastoris

### Example 6: Expression of 2019-nCOV Spike Protein in Human 293 F Cells

The nucleic acid sequence of coronavirus S protein (Wuhan-Hu-1 CoV-S surface bounded protein, which is expressed and bound to the outer surface of 293 F cells) was optimised for expression in human 293 F cells using Geneart for codon optimisation and containing Nhel-NotI single cloning sites to design the nucleic acid sequence of SEQ ID NO: 7.

The codon usage was adapted to the codon bias of Homo sapiens genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content (56%) was adjusted to prolong mRNA half life. Codon usage was adapted to the bias of Homo sapiens resulting in a CAI value of 0.94. The optimized gene has been designed to allow high and stable expression rates in human cells.

### Example 7: Expression of a Fusion Protein Comprising 2019-nCOV Spike Protein in 293 F Cells

The nucleic acid sequence of coronavirus S protein (Wuhan-Hu-1-HBSAg-CoV-S) was optimised for expression in 293 F cells as a fusion protein with Hepatitis B surface antigen. The sequence contains Nhel-NotI single cloning sites to design the nucleic acid sequence of SEQ ID NO: 8.

The codon usage was adapted to the codon bias of Homo sapiens genes. In addition, regions of very high (>80%) or very low (<30%) GC content have been avoided where possible. Negative cis-acting sites (such as splice sites, TATA-boxes, etc.) which may negatively influence expression were eliminated wherever possible. GC content (56%) was adjusted to prolong mRNA half life. Codon usage was adapted to the bias of Homo sapiens resulting in a CAI* (codon adaptation Index) value of 0.94. The optimized gene has been designed to allow high and stable expression rates in human cells.

### Example 8: Adjuvination of 2019-nCOV Spike Protein Using Aluminium Hydroxide and Aluminium Phosphate Gel

The 2019-nCOV spike protein and fusion proteins thereof of Examples 1,3 and 6 were adsorbed in 0.5 mg of aluminium hydroxide and aluminium phosphate gel for adjuvanation.

### Example 9: Adjuvination of 2019-nCOV Spike Protein Using Monophosphoryl Lipid and Aluminium Hydroxide

The 2019-nCOV spike protein and fusion proteins thereof of Examples 1, 3 and 6 were mixed in monophosphoryl lipid (MPL) and aluminium hydroxide for adjuvanation.

### Example 10: Adjuvination of 2019-nCOV Spike Protein Using MF59

The 2019-nCOV spike protein and fusion proteins thereof of Examples 1, 3 and 6 were mixed in MF59 (5% squalene) for adjuvanation.

### Example 11: Generation of an Antibody Response in Mice Immunised with 2019-nCOV Spike Protein

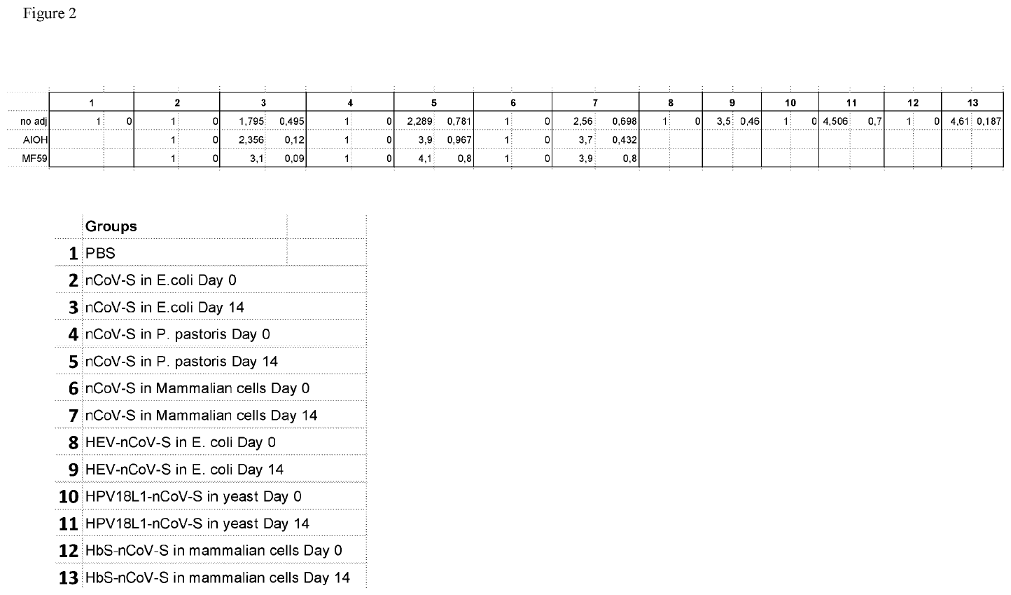

The immunogenicity of formulations comprising the 2019-COV spike proteins and fusion proteins thereof of Examples 2, 4, 5 and 7 without adjuvants and the adjuvanated formulations of Examples 1, 3 and 6 were tested in BALB/c mice (5 mice per group). Vaccination was done on day 0 and 7 with 2 μg antigen per administration. On day 0 and 14 serum samples were tested for sero negativity (day 0) and antibody response against S protein (day 14) by indirect ELISA. All formulations at day 14 induced high antibody titres while adjuvanted ones induced maximum response (FIG. 2).

### Example 12: Expression of HBSAg-(EAAAK)₃-RBD Fusion Protein in HEK Cells (293F)

The HBSAg-(EAAAK)₃-RBD fusion protein of SEQ ID NO: 28 was expressed in HEK cells using the codon-optimised nucleic acid sequence of SEQ ID NO: 27. Briefly, the HBSAg-(EAAAK)₃-RBD gene was optimised for human cell (293F) expression, cloned using Nhe-NotI into pcDNA3.1(+) and subject to clonal selection before transfection into 293F cells in suspension culture. The secreted HBSAg-(EAAAK)₃-RBD fusion protein was harvested after 40h.

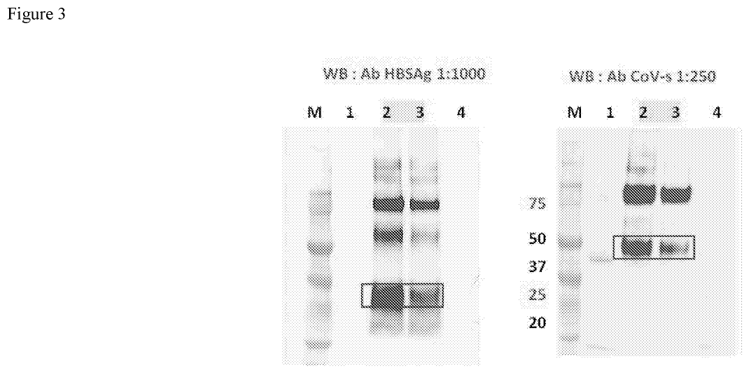

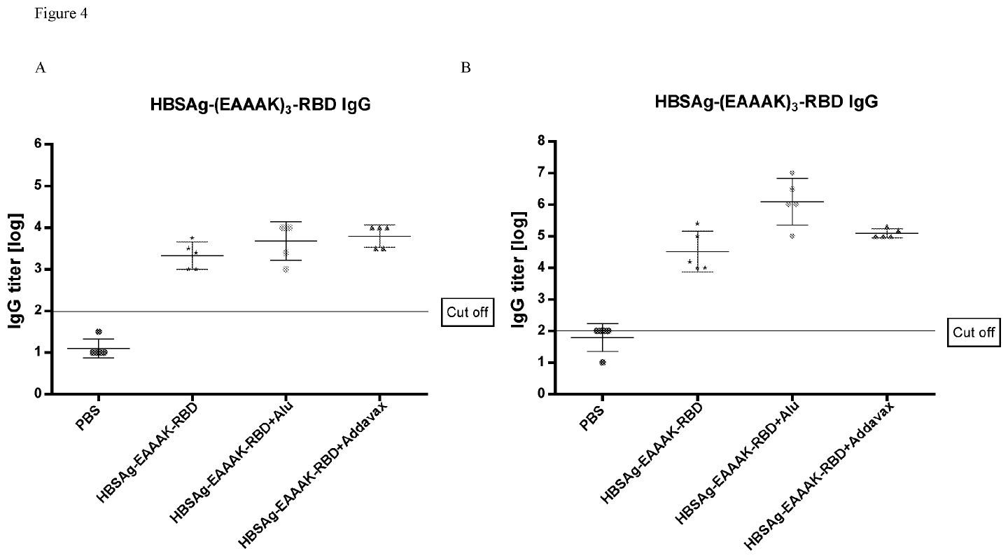

Western blotting using a polyclonal antibody against the 2019-nCOV spike protein (1:250) and a mouse monoclonal antibody against HBSAg (1:1,000) demonstrated strong secretion of the fusion protein by the HEK cells (FIG. 3) in two separate cultures—#2A and #2B.

### Example 13: Generation of an Antibody Response in Mice Immunised with HBSAg-(EAAAK)₃-RBD Fusion in HEK Cells

The HBSAg-(EAAAK)₃-RBD (#2A) from Example 12 was concentrated through 3K Amicon 15 ml centrifugal filter device spinning at 4000 rpm×40 min.

Balb/c mice were then immunised using either (i) 50 μg/dose (100 μl); or (ii) a 50 μg/dose (70 μl), at a 1:1 v/v ratio with either aluminium hydroxide or Addavax™ (total volume administered=140 μl). Mice were primed with the vaccine on d0, followed by a first boost at d7, a second boost at d14 (with bleeding for analysis) and a third boost at d28. The final bleeding for analysis was conducted on d42. The experimental groups are shown in Table 1 below.

TABLE 1
Experimental Groups for mice immunised
with HBSAg-(EAAAK)₃-RBD

| | ANIMAL | | | | |
|---|---|---|---|---|---|
| | No | | | INJECTION | |
| GROUP | Balb/c | CAGE | FORMULATION | VOLUME | |
| 8 | 3 | 5 | Vaccine HBSAg- | 100 | ml (ip) |
| | | | (EAAAK)₃-RBD (293F) | | |
| 9 | 2 | 5 bis | Vaccine HBSAg- | 100 | ml (ip) |
| | | | (EAAAK)₃-RBD (293F) | | |
| 10 | 3 | 6 | Vaccine HBSAg- | 140 | ml (ip) |
| | | | (EAAAK)₃-RBD (293F) | | |
| | | | with adjuvant Alu-280 | | |
| | | | (1:1 v/v ratio) | | |
| 11 | 2 | 6 bis | Vaccine HBSAg- | 140 | ml (ip) |
| | | | (EAAAK)₃-RBD (293F) | | |
| | | | with adjuvant Alu-280 | | |
| | | | (1:1 v/v ratio) | | |
| 12 | 3 | 7 | Vaccine HBSAg- | 140 | ml (ip) |
| | | | (EAAAK)₃-RBD (293F) | | |
| | | | with adjuvant Adda-Vax | | |
| | | | (1:1 v/v ratio) | | |
| 13 | 2 | 7 bis | Vaccine HBSAg- | 140 | ml (ip) |
| | | | (EAAAK)₃-RBD (293F) | | |
| | | | with adjuvant Adda-Vax | | |
| | | | (1:1 v/v ratio) | | |

Antibody titres were assessed at d14 and d42 using ELISA. As shown in FIG. 4, VLPs of HBSAg-(EAAAK)₃-RBD were able to achieve significant IgG titres at d14 and d42, with higher titres observed at d42. Administration with either adjuvant (aluminium hydroxide or AddaVax™) further increased the titre, again as shown in FIG. 4.

Neutralisation assays were also conducted using mice immunized with VLPs of HBSAg-(EAAAK)₃-RBD (alone or with aluminium hydroxide or AddaVax™). The results are shown in Table 2 below. The average neutralising titre was 1:1,200-1:2,700. The highest titre achieved was 1:5,120 and the lowest 1:640.

TABLE 2
Results of neutralisation assay using HBSAg-EAAAK₃-RBD

| TREATMENT | MOUSE | TEST1 | TEST2 |
|---|---|---|---|
| PBS | 1A | 5 | 5 |
| | 1B | 5 | 5 |
| | 1C | 5 | 5 |
| | 1D | 5 | 5 |
| HBSAg-(EAAAK)₃-RBD | 5A | 640 | 320 |
| | 5B | 1280 | 1280 |
| | 5C | 5120 | 5120 |
| | 5BIS A | 2560 | 2560 |
| | 5BIS B | 640 | 640 |
| HBSAg-(EAAAK)₃-RBD + ALU | 6A | 1280 | 2560 |
| | 6B | 2560 | 2560 |
| | 6C | 2560 | 2560 |
| | 6 BIS A | 640 | 640 |
| | 6 BIS B | 2560 | 5120 |
| HBSAg-(EAAAK)₃-RBD + ADDAVAX | 7A | 1280 | 1280 |
| | 7B | 1280 | 1280 |
| | 7C | 640 | 640 |
| | 7 BIS A | 1280 | 1280 |
| | 7 BIS B | 1280 | 2560 |

### Example 14: Expression of HEV-(GGGGS)₃-RBD Fusion in E. coli

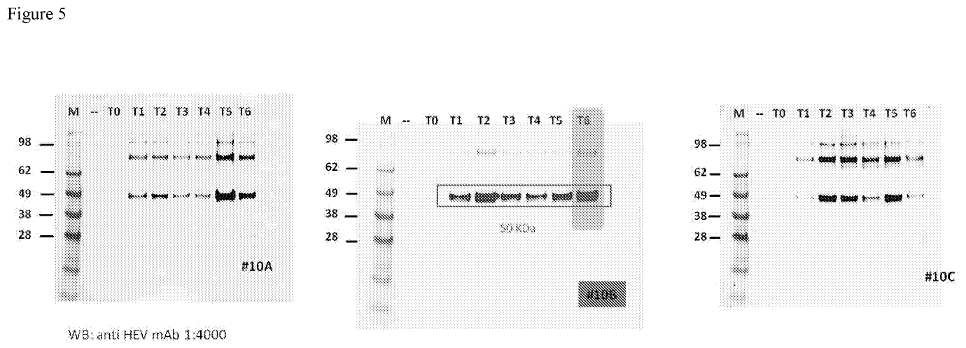

The HEV-(GGGGS)₃-RBD fusion protein of SEQ ID NO: 31was expressed in E. coli using the nucleic acid sequence of SEQ ID NO: 30, which is codon-optimised for expression in E. coli. This nucleic acid sequence was cloned using SacI-NotI into pET26(+). A positive clone was selected (#10) and expressed in BL21. Three different positive colonies (#10A, #10B and #10C) were screened for protein expression, with HEV-RBD expression valuated using IPTG induction (6 hours) for all of #10A, #10B and #10C. Western blotting using an antibody against HEV was used to confirm fusion protein expression in all of #10A, #10B and #10C (FIG. 5). Clone #10B was selected for future investigation and was purified using anion exchange chromatography using a 50 mM phosphate buffer at pH 7.2 as the binding buffer (data not shown).

### Example 15: Generation of an Antibody Response in Mice Immunised with HEV-(GGGGS)₃-RBD Fusion Protein Generated in E. coli

Balb/c mice were immunised using HEV-(GGGGS)₃-RBD (#10B) from Example 14 using 50 μg/dose, at a 1:1 v/v ratio with either aluminium hydroxide or Addavax™ (total volume administered=100 μl). Mice were primed with the vaccine on do, followed by a first boost at d7, a second boost at d14 (with bleeding for analysis) and a third boost at d28. The final bleeding for analysis was conducted on d42. The experimental groups are shown in Table 3 below.

TABLE 3
Experimental Groups for mice immunised
with HEV-(GGGGS)₃-RBD

| | ANIMAL | | | |
|---|---|---|---|---|
| | N* | | | INJECTION |
| GROUP | Balb/c | CAGE | FORMULATION | VOLUME |
| 1 | 4 | 1 | PBS (negative control) | 100 μl (ip) |
| 2 | 3 | 2 | Vaccine HEV-(GGGGS)₃- | 100 μl (ip) |
| | | | RBD (E. coli) | |
| 3 | 2 | 2 bis | Vaccine HEV-(GGGGS)₃- | 100 μl (ip) |
| | | | RBD (E. coli) | |
| 4 | 3 | 3 | Vaccine HEV-(GGGGS)₃- | 100 μl (ip) |
| | | | RBD (E. coli) with | |
| | | | adjuvant ALU-280 | |
| | | | (1:1 v/v ratio) | |
| 5 | 2 | 3 bis | Vaccine HEV-(GGGGS)₃- | 100 μl (ip) |
| | | | RBD (E. coli) with | |
| | | | adjuvant ALU-280 | |
| | | | (1:1 v/v ratio) | |
| 6 | 3 | 4 | Vaccine HEV-(GGGGS)₃- | 100 μl (ip) |
| | | | RBD (E. coli) with | |
| | | | adjuvant Adda-Vax | |
| | | | (1:1 v/v ratio) | |
| 7 | 2 | 4 bis | Vaccine HEV-(GGGGS)₃- | 100 μl (ip) |
| | | | RBD (E. coli) with | |
| | | | adjuvant Adda-Vax | |
| | | | (1:1 v/v ratio) | |

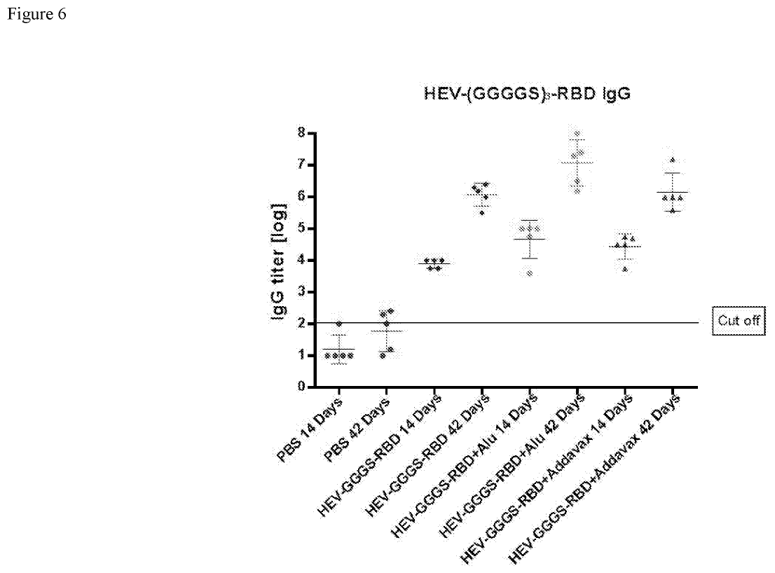

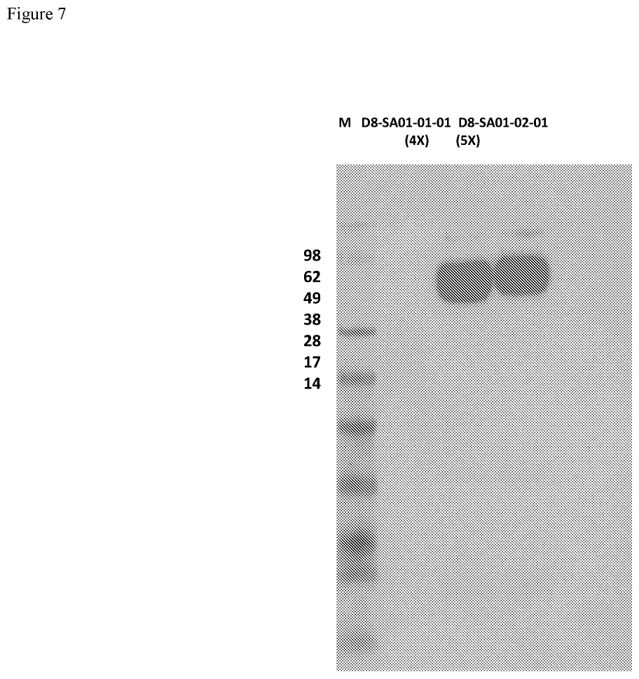

Antibody titres were assessed at d14 and d42 using ELISA. VLPs of HEV-(GGGGS)₃-RBD were able to achieve significant neutralising titres at d14 and d42, with higher titres observed at d42. Administration with either adjuvant (aluminium hydroxide or AddaVax™) further increased the titre, as shown in FIG. 6.

1. An isolated or recombinant polynucleotide encoding a spike protein from 2019-nCOV having at least 90% identity with SEQ ID NO:1, or a spike protein fragment thereof that has a common antigenic cross-reactivity with said spike protein, wherein said polynucleotide is optimised for recombinant expression.

2. The isolated or recombinant polynucleotide of claim 1, which is optimised for expression in a host cell selected from:
(a) Escherichia coli;
(b) yeast, optionally preferably Komagataella or Saccharomyces; and/or
(c) mammalian cells, optionally preferably human cells.

3. The isolated or recombinant polynucleotide of claim 1, wherein one or more cis-acting sequence motif or motifs is omitted from the spike protein, said one or more cis-acting sequence motif or motifs being independently selected from:
(a) an internal TATA-box;
(b) a chi-site;
(c) a ribosomal entry site;
(d) an AT-rich and/or GC-rich stretch of sequence;
(e) an RNA instability motif;
(f) a repeat sequence and/or an RNA secondary structure;
(g) a cryptic splice donor site;
(h) a cryptic splice acceptance site; and/or
(i) any combination of (a) to (h).

4. The isolated or recombinant polynucleotide of claim 1, wherein the polynucleotide is capable of integration into a host cell genome.

5. The isolated or recombinant polynucleotide of claim 1, which has a codon adaptation index (CAI) of at least about 0.80, or at least about 0.9, or at least about 0.93.

6. The isolated or recombinant polynucleotide of claim 5, which comprises or consists of a nucleic acid sequence having:
(a) at least 90% sequence identity to SEQ ID NO: 2;
(b) at least 90% sequence identity to SEQ ID NO: 3;
(c) at least 90% sequence identity to SEQ ID NO: 4;
(d) at least 90% sequence identity to SEQ ID NO: 5;
(e) at least 90% sequence identity to SEQ ID NO: 6;
(f) at least 90% sequence identity to SEQ ID NO: 7;
(g) at least 90% sequence identity to SEQ ID NO: 8;
(h) at least 90% sequence identity to SEQ ID NO: 13;
(i) at least 90% sequence identity to SEQ ID NO: 14;
(j) at least 90% sequence identity to SEQ ID NO: 26;
(k) at least 90% sequence identity to SEQ ID NO: 27;
(l) at least 90% sequence identity to SEQ ID NO: 29;
(m) at least 90% sequence identity to SEQ ID NO: 30; or
(n) at least 90% sequence identity to SEQ ID NO: 32.

7. The isolated or recombinant polynucleotide of claim 1, wherein the encoded spike protein, or fragment thereof:
(a) retains conformational epitopes present in the native 2019-nCOV spike protein;
(b) results in the production of neutralising antibodies specific for the spike protein or fragment thereof when the nucleic acid or the encoded spike protein or fragment thereof is administered to a subject; and/or
(c) comprises or consists of receptor-binding domain (RBD) of the 2019-nCOV spike protein, optionally having at least 90% identity with SEQ ID NO: 15.

8. An expression construct, a viral vector, a RNA vaccine or a DNA plasmid comprising or having contained therein a polynucleotide of claim 1, wherein the polynucleotide is operably linked to a promoter.

9. A vaccine composition comprising a spike protein from 2019-nCOV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, optionally having at least 90% identity with SEQ ID NO: 15,
and optionally the spike protein is capable of generating neutralizing antibodies specific for the spike protein or fragment thereof when administered to a subject.

10. (canceled)

11. A viral vector, RNA vaccine or DNA plasmid that expresses, or comprises a nucleic acid sequence encoding, a spike protein from 2019-nCOV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, optionally preferably having at least 90% identity with SEQ ID NO: 15, and optionally formulated with a pharmaceutically acceptable carrier or diluent.

12. The viral vector, RNA vaccine or DNA plasmid of claim 11, wherein:
(a) the spike protein or fragment thereof further comprises a signal peptide, and optionally the signal peptide directs secretion from a human cell;
(b) the viral vector, RNA vaccine or DNA plasmid further comprises a nucleic acid sequence encoding one or more additional antigens or a fragment thereof, wherein optionally the one or more additional antigens are 2019-nCOV antigens, or a fragment thereof.
and optionally the spike protein or fragment thereof and the one or more additional antigens or fragment thereof are expressed: (i) as a fusion protein; or (ii) separately, encoded in separate viral vectors, RNA vaccines or DNA plasmids for use in combination.

13-16. (canceled)

17. A fusion protein comprising a spike protein from 2019-nCOV having at least 90% identity with SEQ ID NO: 1, or a fragment thereof, that has a common antigenic cross-reactivity with said spike protein, wherein optionally said fragment comprises or consists of receptor-binding domain (RBD) of the 2019-nCOV spike protein, optionally preferably having at least 90% identity with SEQ ID NO: 15.

18. The fusion protein of claim 17, which further comprises:
(a) the Hepatitis B surface antigen, or a fragment thereof that has a common antigenic cross-reactivity with said Hepatitis B surface antigen;
(b) the HPV 18 L1 protein, or a fragment thereof that has a common antigenic cross-reactivity with said HPV 18 L1 protein;
(c) the Hepatitis E P239 protein, or a fragment thereof that has a common antigenic cross-reactivity with said Hepatitis E P239 protein; and/or
(d) the HPV 16 L1 protein, or a fragment thereof that has a common antigenic cross-reactivity with said HPV 16 L1 protein;
wherein optionally:
(i) the fusion protein is encoded by a polynucleotide which comprises or consists of a nucleic acid sequence having at least 90% identity with any one of SEQ ID NO: 3, 5, 6, 8, 26, 27, 29,30, or 32; and/or
(ii) the fusion protein comprises of consists of an amino acid sequence having at least 90% identity with any one of SEQ ID NO: 9, 10, 11, 12, 28, 31, or 33.

19. A virus-like particle (VLP) comprising a spike protein from 2019-nCOV having at least 90% identity with SEQ ID NO: 1, or a fragment thereof, that has a common antigenic cross-reactivity with said spike protein,
wherein optionally said fragment comprises or consists of receptor-binding domain (RBD) of the 2019-nCOV spike protein, optionally having at least 90% identity with SEQ ID NO: 15;
wherein optionally said VLP comprises or consists of a fusion protein as defined in claim 17.

20. An antibody, or binding fragment thereof, that specifically binds to a 2091-nCOV spike protein antigen, or fragment thereof, as defined in claim 1, wherein optionally the antibody is a monoclonal or polyclonal antibody, and optionally the antibody is a Fab, F(ab′)2, Fv, scFv, Fd or dAb.

21-22. (canceled)

23. An oligonucleotide aptamer that specifically binds to a 2019-nCOV spike protein or fragment thereof as defined in claim 1.

24. A vaccine composition comprising the viral vector, and/or RNA vaccine and/or DNA plasmid of claim 11.

25. A method for treating or preventing a 2019-nCoV infection comprising administering to an individual in need thereof a vaccine composition of claim 9.

26. A method for treating or preventing a 2019-nCoV infection comprising administering to an individual in need thereof a vector and/or RNA vaccine and/or DNA plasmid of claim 11.

27. A method of producing a spike protein from 2019-nCOV having at least 90% identity with SEQ ID NO: 1, or a fragment thereof, comprising expressing a polynucleotide as defined in claim 1 in a host cell, and optionally purifying the spike protein or fragment, and optionally the method further comprises formulating said spike protein or fragment thereof with a pharmaceutically acceptable carrier or diluent.

28. (canceled)
